(19) United States
(12) Patent Application Publication
(10) Pub. No.: US 2024/0008971 A1
(43) Pub. Date: Jan. 11, 2024

(54) Ophthalmic Implant

(71) Applicants: Nacuity Pharmaceuticals, Inc., Fort Worth, TX (US); Evonik Corporation, Parsippany, NJ (US)

(72) Inventors: G. Michael Wall, Fort Worth, TX (US); Lianjun Shi, Cumming, GA (US); Troy Carter, Hoover, AL (US); Harsh Patel, Union, KY (US)

(21) Appl. No.: 18/348,811

(22) Filed: Jul. 7, 2023

Related U.S. Application Data
(60) Provisional application No. 63/358,946, filed on Jul. 7, 2022.

(51) Int. Cl. A61F 2/14; A61F 9/00; A61K 31/195; A61K 9/00; A61P 27/12

(52) U.S. Cl. CPC A61F 2/14; A61F 9/0017; A61K 31/195; A61K 9/0051; A61P 27/12

(57) ABSTRACT

The present invention relates in general to compositions and methods for treating an animal or human patient in need of treatment for ophthalmic diseases or conditions involving oxidative stress and treatment with an ophthalmic implant containing (2R,2R′)-3,3′-disulfanediyl bis(2-cetamidopropanamide) (diNACA), wherein such ophthalmic diseases or conditions include but not limited to cataracts, cataracts in a subject that does not have diabetes, corneal endothelial cell loss, age-related macular degeneration, presbyopia, retinitis pigmentosa (RP), Usher syndrome, Stargardt syndrome, glaucoma, diabetic retinopathy, and/or retinal disease.

### CROSS-REFERENCE TO RELATED APPLICATIONS

This application claims priority to U.S. Provisional Application Ser. No. 63,358,946, filed Jul. 7, 2022, the entire contents of which are incorporated herein by reference.

### STATEMENT OF FEDERALLY FUNDED RESEARCH

None.

### TECHNICAL FIELD OF THE INVENTION

The present invention relates in general to identifying an animal or human patient in need of treatment for ophthalmic diseases or conditions involving oxidative stress and treatment with an ophthalmic implant containing (2R,2R′)-3,3′-disulfanediyl bis(2-acetamidopropanamide), (diNACA) such ophthalmic diseases or conditions including but not limited to cataracts, cataracts in a subject that does not have diabetes, corneal endothelial cell loss, age-related macular degeneration, presbyopia, retinitis pigmentosa (RP), Usher syndrome, Stargardt syndrome, glaucoma, diabetic retinopathy and/or other retinal disease.

### BACKGROUND OF THE INVENTION

Age-related macular degeneration (AMD) is the term used for describing lost or blurred vision in the center of the visual field. A difficulty with detecting early stages of AMD is the lack of symptoms, which are very gradual and may affect one or both eyes. While all vision may not be lost, the loss of vision in the center of the visual field makes it difficult to recognize objects, drive, read, or perform normal activities. Typically, AMD is a disease that affects individuals later in life, and is made worse by smoking, hypertension, atherosclerosis, elevated cholesterol, obesity, fat intake, and exposure to sunlight. There are also genetic components to AMD, as sibling studies have shown an increase in recurrence ratios, and, to date, at least 5 different genes have shown some linkage to AMD. Unfortunately, AMD is a complex disease that results from a variety of environmental, genetic, and lifestyle factors.

Clinical signs of AMD include a distortion in the visual field, which typically takes the form of metamorphopsia, which is a type of vision distortion in which a grid of straight lines appears wavy and parts of the grid may appear blank. Other symptoms of AMD include: slow recovery of visual function as a result of exposure to bright light (e.g., using a photo-stress test), a drastic decrease in visual acuity, blurred vision, trouble discerning colors, and a loss of contrast sensitivity. AMD shows a processor accumulation of drusen deposits in the macula, between the retinal pigment epithelium, and the underlying choroid. These drusen are the build-up of extracellular proteins and lipids that are believed to damage the retina over time. However, the presence of drusen is not indicative of disease progression, as the majority of people over age 60 have drusen without any negative effects. Various stages of AMD are known, and are generally divided into early AMD, intermediate AMD, late AMD, Dry (or nonexudative) AMD, atrophic (or geographic) AMD, and/or wet (or exudative) AMD. Currently, there are drug products approved to treat “wet” AMD but not “dry” AMD.

Diabetic retinopathy (DR) is a leading cause of blindness globally and its pathogenesis has still not been completely elucidated. Some studies show a close relation between oxidative stress. Thu et al. (2012) conducted a study aimed to investigate the effects of anti-oxidant in DR and expression of vascular endothelial growth factor (VEGF) and intercellular adhesion molecule-1 (ICAM-1) from retinal blood vessels in diabetic rats. Diabetic rat models were established by intraperitoneal injection of streptozotocin (60 mg/kg) and confirmation of high serum glucose levels in the animals. Antioxidant N-acetylcysteine was given to diabetic rats to elicit antioxidative responses, and rats were sacrificed at 3 and 5 months. Ultrastructures of retinal vascular tissues were observed under transmission electron microscope, and pathology of retinal capillaries was examined using retinal vascular digest preparations. Changes in the expression of VEGF and ICAM-1 were examined by immunofluorescence; and reactive oxygen species contents in the retinas were detected using dichlorofluorescein assay. Compared with normal rats, diabetic rats displayed significant retinopathy both under electronic and light microscopy, accompanied by elevated reactive oxygen species contents in the retinas; N-acetylcysteine treatment alleviated the pathological changes and also decreased reactive oxygen species, most significantly at 5 months. VEGF and ICAM-1 expressions were significantly up-regulated in retinal blood vessels from diabetic rats, and such up-regulation was attenuated by N-acetylcysteine treatment. The expression of both factors returned to basal levels after 5-month treatment with N-acetylcysteine. Long-term N-acetylcysteine treatment exerts protective effects on the diabetic retinas, possibly through its down-regulation of the expression of VEGF and ICAM-1, and reduction of reactive oxygen species content in retinal vascular tissues in diabetic rats. (Thu et al. 2012).

Vitreoretinal eye surgery includes a group of procedures performed deep inside the eye with lasers or conventional surgical instruments. The goal of vitreoretinal surgery is to preserve, restore, and enhance vision for individuals suffering from one of a range of blinding conditions. The incidence of vitreoretinal surgery is about 225,000 annually in the U.S. Despite the effectiveness of vitreoretinal surgery, it is associated with various undesirable complications. By far, the most prevalent complication is cataract formation (cloudiness or opacity of the crystalline lens that focuses light onto the retina). Following vitreoretinal surgery, significant cataracts occur in 80% of patients within the first year (Belin and Parke, 2020).

The importance of the vitreous in protecting the lens from oxygen and from nuclear cataract was highlighted by studies in which the vitreous gel was intentionally preserved or destroyed. Surgery to remove preretinal membranes typically involves vitrectomy, which is soon followed by nuclear cataract. However, patients who had a variation of the usual procedure, which did not destroy the vitreous gel, did not develop nuclear cataracts, even after a 5-year follow-up. These studies were important because they showed that nuclear cataract could be prevented, even in patients who had surgery to repair a retinal problem, by simply preserving the structure of the vitreous. Unfortunately, patients who had this modified procedure were more likely to have recurrence of the preretinal fibrous membranes, obviating the lens-sparing nature of the modified surgery. Recent studies in animals showed that liquefying the vitreous or creating an enzyme-induced detachment of the vitreous from the retina increased the level of oxygen in the vitreous and resulted in higher levels of oxygen reaching the lens nucleus. These studies demonstrate that the gel state of the vitreous protects the lens from oxygen and from nuclear cataract. The liquefaction of the vitreous, which occurs to a varying extent with age, might be considered to be a slow form of vitrectomy. Loss of the gel state of the vitreous is likely to increase the exposure of the lens to oxygen and place patients at increased risk for nuclear cataract (Beebe et al., 2010).

A safe, effective pharmacotherapy that prevents cataract formation arising from vitreoretinal surgery would be a medical breakthrough, obviating the need for subsequent cataract surgery. Accumulating experimental evidence indicates that oxidative stress is a factor in development of cataracts. (Lim et al., 2016). Therefore, an agent that treats oxidative stress should have activity as an anticataract agent.

Glaucoma describes several eye diseases that result from damage to the optic nerve leading to loss of vision. Primary Open Angle Glaucoma (POAG) is a chronic, irreversible optic neuropathy leading to the progressive death of retinal ganglion cells, clinically observed as silent visual field loss along with a decrease in color and contrast sensitivity (Ster et al., 2014). POAG is the leading causes of irreversible blindness globally (1) yet our approaches to its treatment have remained essentially unchanged for over 100 years: eye drops to lower intraocular pressure (TOP) as an initial approach and surgical enhancement of outflow if eye drops are not sufficient. An increase in IOP is a major risk factor for glaucoma, as are a family history of glaucoma and high blood pressure, however, the etiology of glaucoma is still under investigation. While elevated IOP is a major risk factor (perhaps even the most important singular factor) in relation to glaucoma neuropathy, it is by no means the only factor responsible for glaucoma neuropathy. Extensive investigations have revealed multiple additional risk factors such as mutations in specific nuclear genes, increased glutamate levels, alteration in nitric oxide (NO) metabolism, changes in the mitochondrial genome, vascular disturbances, and toxic effects and oxidative damage caused by reactive oxygen species (ROS) (Ster et al., 2014). The pathogenic role of ROS in glaucoma is supported by various experimental findings, including (a) resistance to aqueous humor outflow is increased by hydrogen peroxide by inducing trabecular meshwork (TM) degeneration; (b) TM possesses remarkable antioxidant activities, mainly related to superoxide dismutase-catalase and glutathione pathways that are altered in glaucoma patients; and (c) IOP increase and severity of visual-field defects in glaucoma patients parallel the amount of oxidative DNA damage affecting TM (Izzotti et al., 2006).

Cataracts are any opacification of the lens. Cataracts are often considered to be an unavoidable consequence of aging. Age-related cataracts are responsible for nearly half of all blindness worldwide and half of all visual impairment in the USA. In the USA alone, surgery for age-related cataracts is the most expensive ocular surgical procedure, with expenditures exceeding USD 3 billion annually. Lens opacities may result from protein aggregation, protein phase separation, or disturbance of the regular alignment or packing of the fiber cells, all of which may lead to increased light scattering. Nuclear opacities sometimes also involve increased coloration, resulting in decreased light transmission. Oxidative damage is a major cause or consequence of cortical and nuclear cataracts, the most common types of age-related cataract. The preponderance of evidence suggests that exposure to increased levels of molecular oxygen accelerates the age-related opacification of the lens nucleus, leading to nuclear cataract. Factors in the eye that maintain low oxygen partial pressure around the lens are, therefore, important in protecting the lens from nuclear cataract (Beebe et al., 2010).

The vitreous body is a clear, gel-like substance that fills the cavity of the eye behind the lens and helps to stabilize the various retinal layers and retinal vasculature. The gel-like characteristics of the vitreous body result from a network of collagen fibrils that extend throughout the gel. Because the vitreous gel has no circulatory flow and does not mix, it effectively impedes the distribution of signaling molecules or nutrients that may be released from surrounding tissues.

Although the vitreous gel is remarkably stable, over time there is a gradual tendency for the gel to collapse, likely a result of degradation or alteration of the collagen fiber network. Patients with degenerate vitreous bodies are at increased risk for tractional forces to develop on the underlying retina, which predisposes them to retinal detachment and disruption of the delicate laminations that characterize the healthy retina. Apart from age-related vitreous liquefaction, other conditions can require removal of the vitreous gel, such as uncontrolled hemorrhaging from unstable retinal blood vessels, typical of patients with diabetic retinopathy. In such conditions, a vitrectomy procedure is used to remove the natural gel along with entrapped blood components followed by replacement with a balanced salt solution. Although vitrectomy is a major therapeutic advance in the treatment of retinal disease, it carries with it a high risk for nuclear cataract development. Recent studies point to oxygen as a cataract-inducing by-product of the vitrectomy procedure. Most oxygen in the vitreous gel is thought to originate from the retinal vasculature. Because of its diffusion-limiting physical properties, an oxygen gradient exists in the native eye, with highest levels nearest the retina and comparatively lower levels farthest away near the posterior surface of the lens. When the contents of the vitreous humor are removed by vitrectomy and replaced with a saline solution, the physical barrier to oxygen diffusion is destroyed. Because components in the vitreous replacement solution mix freely, small molecules released from retinal vessels are distributed throughout the vitreous cavity, thus exposing the posterior lens to abnormally high levels of oxygen (Petrash, 2013).

Since vitrectomy surgery is a common procedure that causes rapid and reliable opacification of the lens nucleus, patients undergoing vitrectomy offer a promising ‘model system’ to test an anticataract hypothesis. Success in protecting post-vitrectomy patients from developing nuclear cataracts would lay the groundwork for interventions to prevent these cataracts in subjects with advanced vitreous degeneration (Beebe et al., 2010).

Presbyopia is the condition whereby the ability to focus on near objects becomes diminished. The impact of presbyopia is nearly universal among people as they enter their middle-age years. Although near vision can be regained simply by the use of reading spectacles, for practical and professional reasons there is great demand for a remedy that does not require corrective lenses. The biological basis for presbyopia has received intense study over the years. To adjust for near vision, it is necessary for the lens to “round up” from its unaccommodated shape. This shape change is made possible when the ciliary muscles contract, drawing together the tissues that surround the lens in a sphincter-like motion. In response, the body of the lens assumes a more rounded shape, altering the radius of curvature mainly at the anterior surfaces. The accommodation response reduces the focal length of the lens to allow clear focusing of near objects on the retina. Thus, the process of accommodation for near vision relies on a change in lens shape in response to contraction of the ciliary muscles (Petrash, 2013).

As the lens ages, the nucleus becomes much stiffer, but the cortex remains soft. Stiffening of the nucleus is a major contributor to presbyopia (Beebe et al., 2010).

Pliability is a critical factor that underlies the ability of the lens to change shape. Many investigators have shown that the human lens hardens over time, leading to the hypothesis that presbyopia occurs when the lens becomes too hard to change shape in response to ciliary muscle contraction. Studies by a variety of methods have demonstrated a marked increase in lens stiffness beginning in the third to fifth decade of life, which is roughly the age when loss of accommodation becomes noticeable in the early stages of presbyopia. The molecular basis for increased lens stiffness is not well understood. Fiber cells in the lens nucleus are held together by an array of intercellular adhesion molecules, gap junction complexes, and ball-and-socket-like interdigitations along their extensive lateral membranes. As the lens ages and more of the lens mass becomes internalized into the nuclear region, it is likely that cells are riveted together with these intercellular junctions, perhaps contributing to the diminished deformability of the tissue (Petrash, 2013).

The onset of presbyopia in middle adulthood results in potential losses in productivity among otherwise healthy adults if uncorrected or under corrected. There were an estimated 1.272 billion cases of presbyopia worldwide in 2011. A total of 244 million cases, uncorrected or undercorrected among people aged <50 years, were associated with a potential productivity loss of US $11.023 billion (0.016% of global GDP) or, assuming people aged <65 years were productive, the potential productivity loss would be US $25.367 billion (0.037% of global GDP). The economic burden is even more significant in lower-income countries, where up to 94% of cases may be uncorrected or under corrected (Frick et al., 2015).

Retinitis pigmentosa (RP) is a group of diseases in which one of a large number of mutations causes death of rod photoreceptors. After rods die, cone photoreceptors slowly degenerate in a characteristic pattern. The mechanism of rod cell death varies depending upon the gene that is mutated and the rate that rods degenerate is an important prognostic feature, because cones do not begin to degenerate until almost all rods have been eliminated. Rod cell death causes night blindness, but visual disability and blindness result from cone degeneration and therefore it is critical to determine the mechanisms by which it occurs. The death of rods reduces oxygen consumption resulting in high tissue levels of oxygen in the outer retina. The excess oxygen stimulates superoxide radical production by mismatches in the electron transport chain in mitochondria and by stimulation of NADPH oxidase activity in cytoplasm. The high levels of superoxide radicals overwhelm the antioxidant defense system and generate more reactive species including peroxynitrite which is extremely damaging and difficult to detoxify. This results in progressive oxidative damage in cones which contributes to cone cell death and loss of function because drugs or gene transfer that reduce oxidative stress promote cone survival and maintenance of function. Compared with aqueous humor samples from control patients, those from patients with RP show significant elevation of carbonyl content on proteins indicating oxidative damage and a reduction in the ratio of reduced to oxidized glutathione indicating depletion of a major component of the antioxidant defense system from ongoing oxidative stress. The first step in clinical trials will be to identify doses of therapeutic agents that reverse these biomarkers of disease to assist in design of much longer trials with functional and anatomic endpoints (Campochiaro and Mir, 2018).

N-acetylcysteine (NAC) is a free radical scavenger that is converted to cysteine, the substrate for synthesis of glutathione, a key component of the endogenous antioxidant defense system (Dilger and Baker, 2007). NAC is approved for treatment of acetaminophen overdose and is a life-saving treatment if administration is begun relatively soon after ingestion; this is quite remarkable since in untreated patients the severity of oxidative damage in liver is so severe that widespread liver necrosis and liver failure occur (Prescott et al., 1977; Smilkstein et al., 1988). Compared to untreated rd10 mice, those given NAC in drinking water showed dose-dependent, statistically significant reduction in oxidative damage and improvements in cone survival and function (Lee et al., 2011). Over many months of NAC treatment, cone function and survival showed some deterioration but were still significantly greater than that in untreated mice demonstrating a prolonged effect. N-acetylcysteine amide (NACA) is a precursor of NAC which penetrates the blood-retinal barrier and enters cells better than NAC. Compared to rd10 mice treated with NAC, those treated with a 7-fold lower dose of N-acetylcysteine amide (NACA), showed greater preservation of cone function and survival. (Campochiaro and Mir, 2018).

Although the Argis II Retinal Prosthesis System was approved by FDA in 2013 as an implanted device to treat adults with severe vision loss, it only produces the sensation of light, thereby helping patients identify the location or movement of objects and people; the devise is not disease modifying.

Currently, there is one FDA-approved gene therapy for RP, LUXTURNA, an adeno-associated virus vector-based gene therapy indicated for the treatment of patients with confirmed biallelic RPE65 mutation-associated retinal dystrophy (LUXTURNA Prescribing Information, 2017). Other than LUXTURNA for patients with the RPE65 mutation (biallelic RPE65 mutation-associated retinal dystrophy affects approximately 1,000 to 2,000 patients in the U.S. (FDA, 2017)), there is no other FDA-approved therapy that stops the evolution of the disease or restores vision caused by retinitis pigmentosa. The current therapeutic approach is restricted to slowing down the degenerative process by sunlight protection and vitamin A supplementation, treating complications (cataracts and macular edema), and helping patients to cope with the social and psychological impact of blindness.

U.S. patent application Ser. No. 15/523,665 teaches the use of NACA for the treatment of retinitis pigmentosa. NACA has been shown to improve, to a greater extent than NAC, visual parameters in a mouse model of RP.

As such, there still exists a need for novel compositions and methods for treatment of ophthalmic diseases and conditions related to oxidative stress, including but not limited to, age-related macular degeneration, cataract, corneal endothelial cell protection, glaucoma, diabetic retinopathy, presbyopia, retinitis pigmentosa and other retinal diseases.

### SUMMARY OF THE INVENTION

As embodied and broadly described herein, an aspect of the present disclosure relates to a biodegradable intraocular implant, comprising: (2R,2R′)-3,3′-disulfanediyl bis(2-acetamidopropanamide) (diNACA) in a pharmaceutically acceptable polymer. In one aspect, the implant further comprises an antioxidant selected from the group consisting of N-acetylcysteine, N-acetylcysteine amide, lipoic acid, lipoic acid choline ester, salts thereof, and mixtures thereof. In another aspect, the implant is about 0.1-0.5 mm in diameter and about 4-mm long. In another aspect, the implant is about 0.2-0.4 mm in diameter and about 5-8 mm long. In another aspect, the implant is about 0.3 mm in diameter and about 6-7 mm long. In another aspect, the implant is about 0.4 mm in diameter and about 7 mm long. In another aspect, the implant is between or about 0.05 mm in diameter and between or about 5 to 8 mm long. In another aspect, the biodegradable polymer is a poly (lactide-co-glycolide) copolymer. In another aspect, the diNACA is in the implant at an amount of at least between 21-65 weight percent (wt %), between 1-20 wt %, between 5-15 wt %, between 7-12 wt %, or about 10 wt %. In another aspect, the diNACA is present with an auxiliary agent. In another aspect, the implant comprises a plurality of openings or holes in the polymer. In another aspect, the intraocular implant comprises: an diNACA in the biodegradable polymer in the form of a first intraocular implant; and an auxiliary agent associated with the biodegradable polymer in the form of a second intraocular implant; wherein the diNACA is present in the first intraocular implant in an amount of at least about 10 weight percent. In another aspect, the implant is suitable for intravitreal placement in an eye of an individual, the diNACA being present in an amount effective to treat an ocular condition of the eye; and an auxiliary agent associated with the biodegradable polymeric component in the form of an intraocular implant structured for intravitreal placement in an eye of an individual, the auxiliary agent being present in an amount effective to reduce the occurrence of at least one undesired effect present upon otherwise identical administration of the diNACA alone; wherein the diNACA is present in the intraocular implant in an amount of at least about 10 weight percent. In another aspect, the polymer in the diNACA implant decreases in molecular weight, for instance through hydrolysis, becomes a hydrogel, and then dissolves as evidenced by loss mass to the implant over time. The loss-of-molecular-weight process, controls the release of drug from the implant.

As embodied and broadly described herein, an aspect of the present disclosure relates to a biodegradable intraocular implant system containing an antioxidant suitable for the treatment of ophthalmic diseases or conditions involving oxidative stress in an animal or human. In one aspect, the antioxidant comprises (2R,2R′)-3,3′-disulfanediyl bis(2-acetamidopropanamide) (diNACA) or the combination of (2R,2R′)-3,3′-disulfanediyl bis(2-acetamidopropanamide) (diNACA) and an auxiliary agent in the matrix of a polymer or in alternating layers. In another aspect, the antioxidant comprises (2R,2R′)-3,3′-disulfanediyl bis(2-acetamidopropanamide) (diNACA) or the idNACA and an auxiliary agent are comprised in the same implant or in different implants. In another aspect, the diNACA is in the implant at an amount of at least between 21-65 weight percent (wt %), between 1-20 wt %, between 5-wt %, between 7-12 wt %, or about 10 wt %. In another aspect, the polymer in the diNACA implant decreases in molecular weight, for instance through hydrolysis, becomes a hydrogel, and then dissolves as evidenced by loss mass to the implant over time. The loss-of-molecular-weight process, controls the release of drug from the implant.

As embodied and broadly described herein, an aspect of the present disclosure relates

to a method for treatment of ophthalmic oxidative stress in an animal or human subject that comprises: identifying an animal or human patient in need of treatment for ophthalmic oxidative stress of the eye; and administering to the animal or human patient a therapeutically effective amount of N-acetylcysteine amide (NACA) or (2R,2R′)-3,3′-disulfanediyl bis(2-acetamidopropanamide) (diNACA) in an intravitreal implant (diNACA implant). In one aspect, the ophthalmic oxidative stress of the eye is selected from at least one of cataracts, cataracts in a subject that does not have diabetes, corneal endothelial cell loss, age-related macular degeneration, presbyopia, retinitis pigmentosa (RP), Usher syndrome, Stargardt syndrome, glaucoma, diabetic retinopathy, or retinal disease. In another aspect, the diNACA is provided in or with a pharmaceutically acceptable carrier. In another aspect, the diNACA is in a dosage form for a delivery route selected from the group consisting of intravitreal, intrastromal, intracameral, subtenon, retinal, subretinal, retrobulbar, peribulbar, suprachoroidal, subchoroidal, conjunctival, subconjunctival, episcleral, posterior juxtascleral, anterior juxtascleral, circumcorneal, topical, and tear duct. In another aspect, the diNACA is administered in daily doses of about 0.25, 1, 2, 3, 4, 5, 6, 7, 8, 9, 10, 20, 25, 30, 40, 45, 50, 60, 70, 75, 80, 90, 100, 110, 120, 125, 130, 140 to 150 mg/Kg. In another aspect, the diNACA is administered in a single dose of about 1, 2, 3, 4, 5, 6, 7, 8, 9, 10, 20, 25, 30, 40, 45, 50, 60, 70, 80, 90, 100, 110, 120, 125, 130, 140, 150, 175, 200, 250, 300, 400, 500, 600, 700, 750, 800 to 900 micrograms. In another aspect, the diNACA is administered once every 1, 2, 3, 4, 5, or 6 months. In another aspect, the diNACA is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, NAC, NACA, propyl gallate, α-tocopherol, citric acid, ethylenediamine tetraacetic acid (EDTA), sorbitol, tartaric acid, or phosphoric acid. In another aspect, the diNACA is in the implant at an amount of at least between 21-65 weight percent (wt %), between 1-20 wt %, between 5-15 wt %, between 7-12 wt %, or about 10 wt %. In another aspect, the dose for administration is about 1, 10, 100, 150, 150, 300, 333, 400, 500, 600, 700, 750, 800, 900, 1,000, 2,500, 5,000, 7,500, or 10,000 micrograms per dose. In another aspect, the dose for administration is 100, 150, 150, 300, 333, 400, 500, 600, 700, 750, 800, 900, 1,000, 2,500, 5,000, 7,500, or 10,000 mg per dose. In another aspect, the diNACA implant is manually inserted into the eye. In another aspect, the diNACA implant is injected into the eye. In another aspect, the diNACA implant breaks apart and dissolves or biodegrades in the eye. In another aspect, the polymer in the diNACA implant decreases in molecular weight, for instance through hydrolysis, becomes a hydrogel, and then dissolves as evidenced by loss mass to the implant over time. The loss-of-molecular-weight process, controls the release of drug from the implant. In another aspect, the diNACA is administered prophylactically to prevent ophthalmic disease or condition involving oxidative stress.

As embodied and broadly described herein, an aspect of the present disclosure relates to a nonbiodegradable, nonbioerodible or nonbioabsorbable intraocular implant, comprising: (2R,2R′)-3,3′-disulfanediyl bis(2-acetamidopropanamide) (diNACA) in a pharmaceutically acceptable polymer. In one aspect, the antioxidant is selected from the group consisting of N-acetylcysteine, N-acetyl cy steine amide, (2R,2R′)-3,3′-disulfanediyl bis(2-acetamidopropanamide) (diNACA), lipoic acid, lipoic acid choline ester, salts thereof, and mixtures thereof. In another aspect, the implant is about 0.1-0.5 mm in diameter and about 4-10 mm long. In another aspect, the implant is about 0.2-0.4 mm in diameter and about 5-8 mm long. In another aspect, the implant is about 0.3 mm in diameter and about 6-7 mm long. In another aspect, the implant is about 0.4 mm in diameter and about 7 mm long. In another aspect, the implant is between or about 0.05 mm in diameter and between or about 5 to 8 mm long. In another aspect, the biodegradable polymer is a polycarbamate polyurea, poly(vinyl ester), poly(methyl methacrylate), poly(vinyl chloride), polyamide, nylon, poly(ethylene terephthalate), rubber, silicone, polyisoprene, polyisobutylene, polybutadiene, polyethylene, polytetrafluoroethylene, poly(vinylidene chloride), polyacrylonitrile, polyvinylpyrrolidone) chlorinated polyethylene, polytrifluorochloroethylene, poly(ethylene chlorotrifluoroethylene), polytetrafluoroethylene, poly(ethylene tetrafluoroethylene), poly(4,4-isopropylidene diphenylene carbonate), polyurethane, polyperfluoroalkoxy, poly(vinylidene fluoride), vinylidene chloride-acrylonitrile copolymer, vinyl chloride-diethyl fumarate copolymer, silicone, silicone rubber, polydimethylsiloxanes, ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefin), poly(styrene), poly(halo-olefin), poly(vinyl) ester, alkylacrylate, polyoxides, polyesters, polyamides, and polycarbonates, or mixtures thereof. In another aspect, the diNACA is in the implant at an amount of at least between 21-65 weight percent (wt %), between 1-20 wt %, between 5-15 wt %, between 7-12 wt %, or about 10 wt %. In another aspect, the polymer in the diNACA implant decreases in molecular weight, for instance through hydrolysis, becomes a hydrogel, and then dissolves as evidenced by loss mass to the implant over time. The loss-of-molecular-weight process, controls the release of drug from the implant. In another aspect, the implant further comprises an auxiliary agent. In another aspect, the implant comprises a plurality of openings or holes in the polymer or a coating. In another aspect, the diNACA implant is adapted for administration to the eye at a location selected from the group consisting of intravitreal, intrastromal, intracameral, subtenon, retinal, subretinal, retrobulbar, peribulbar, suprachoroidal, subchoroidal, conjunctival, subconjunctival, episcleral, posterior juxtascleral, anterior juxtascleral, circumcorneal, topical, and tear duct.

As embodied and broadly described herein, an aspect of the present disclosure relates to a method for treatment of a disease or condition of ophthalmic oxidative stress in an eye of an animal or human subject comprising: obtaining an intravitreal implant (diNACA implant) comprising a therapeutically effective amount of N-acetylcysteine amide (NACA) or (2R,2R′)-3,3′-disulfanediyl bis(2-acetamidopropanamide) (diNACA); and delivering to a pre-determined area in, at, or about the eye the diNACA implant that delivers a therapeutically effective amount of the diNACA to the pre-determined area of the eye. In one aspect, the disease or condition is selected from at least one of cataracts, cataracts in a subject that does not have diabetes, corneal endothelial cell loss, age-related macular degeneration, presbyopia, retinitis pigmentosa (RP), Usher syndrome, Stargardt syndrome, glaucoma, diabetic retinopathy, or retinal disease. In another aspect, the diNACA implant is administered to the pre-determined area of the eye selected from the group consisting of intravitreal, intrastromal, intracameral, subtenon, retinal, subretinal, retrobulbar, peribulbar, suprachoroidal, subchoroidal, conjunctival, subconjunctival, episcleral, posterior juxtascleral, anterior juxtascleral, circumcorneal, topical, and tear duct. In another aspect, the diNACA is administered in daily doses of about 0.25 to 150 mg/Kg. In another aspect, the diNACA is administered in a single dose of about 1 to 900 micrograms. In another aspect, the diNACA is administered once every 1, 2, 3, 4, 5, or 6 months. In another aspect, the diNACA is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, NAC, NACA, propyl gallate, α-tocopherol, citric acid, ethylenediaminetetraacetic acid (EDTA), sorbitol, tartaric acid, or phosphoric acid. In another aspect, the diNACA is in the implant at an amount of at least between 21-65 weight percent (wt %), between 1-20 wt %, between 5-15 wt %, between 7-12 wt %, or about 10 wt %. In another aspect, the dose for administration is about 1, 10, 100, 150, 150, 300, 333, 400, 500, 600, 700, 750, 800, 900, 1,000, 2,500, 5,000, 7,500, or micrograms per dose. In another aspect, the dose for administration is 100, 150, 150, 300, 333, 400, 500, 600, 700, 750, 800, 900, 1,000, 2,500, 5,000, 7,500, or 10,000 mg per dose. In another aspect, the diNACA implant is manually inserted into the eye. In another aspect, the diNACA implant breaks apart and dissolves or biodegrades in the pre-determined area of the eye. In another aspect, the polymer in the diNACA implant decreases in molecular weight, for instance through hydrolysis, becomes a hydrogel, and then dissolves as evidenced by loss mass to the implant over time. The loss-of-molecular-weight process, controls the release of drug from the implant. In another aspect, the diNACA is administered prophylactically to prevent ophthalmic disease or condition in or about the pre-determined area of the eye.

### BRIEF DESCRIPTION OF THE DRAWINGS

For a more complete understanding of the features and advantages of the present invention, reference is now made to the detailed description of the invention along with the accompanying figures and in which:

FIGS. 1A, 1B show effects of pre-treatment of NACA (FIG. 1A) and diNACA (FIG. 1B), alone, on rat lens transparency (opacities are marked by *).

FIGS. 2A to 2L show effects of NACA and diNACA on GO-induced cataract in isolated rat lens.

FIG. 3 shows effects of NACA and diNACA inhibition of H₂O₂-induced cataract in isolated porcine lens.

FIG. 4 shows diNACA inhibits cataract formation (opacity) induced by oxidative reagents in ex vivo rat lens and ex vivo porcine lens based on comparison to negative control (no oxidative reagent, clear).

FIG. 5 shows diNACA (NPI-002) Intravitreal Implant Delivers diNACA, NACA and NAC to Rabbit Vitreous Humor.

FIG. 6 show in vitro release profiles of diNACA (NPI-002) intravitreal implants.

FIG. 7 shows process flow for manufacture of implant.

FIG. 8 shows dimensions of implant.

FIG. 9A is a graph that shows the NAC concentration in aqueous humor and vitreous humor at 3 months after surgery in group 1 (control), group 2 (NPI-002, one implant), and group 3 (NPI-002, two implants). Data represent mean±SEM, where n=5-6.

FIG. 9B is a graph that shows the NAC concentration in lens, retina and RPE/choroid at 3 months after surgery in group 1 (control), group 2 (NPI-002, one implant), and group 3 (NPI-002, two implants). Data represent mean±SEM, where n=6.

FIG. 10A is a graph that shows the NACA concentration in aqueous humor and vitreous humor at 3 months after surgery in group 1 (control), group 2 (NPI-002, one implant), and group 3 (NPI-002, two implants). Data represent mean±SEM, where n=5-6.

FIG. 10B is a graph that shows the NACA concentration in lens, retina and RPE/choroid at 3 months after surgery in group 1 (control), group 2 (NPI-002, one implant), and group 3 (NPI-002, two implants). Data represent mean±SEM, where n=6.

FIG. 11A is a graph that shows the di-NACA concentration in aqueous humor and vitreous humor at 3 months after surgery in group 1 (control), group 2 (NPI-002, one implant), and group 3 (NPI-002, two implants). Data represent mean±SEM, where n=5-6.

FIG. 11B is a graph that shows the di-NACA concentration in lens, retina and RPE/choroid at 3 months after surgery in group 1 (control), group 2 (NPI-002, one implant), and group 3 (NPI-002, two implants). Data represent mean±SEM, where n=6.

FIG. 12A is a graph that shows the GSH concentration in aqueous humor and vitreous humor at 3 months after surgery in group 1 (control), group 2 (NPI-002, one implant), and group 3 (NPI-002, two implants). Data represent mean±SEM, where n=5-6.

FIG. 12B is a graph that shows the GSH concentration in lens, retina and RPE/choroid at 3 months after surgery in group 1 (control), group 2 (NPI-002, one implant), and group 3 (NPI-002, two implants). Data represent mean±SEM, where n=6.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, weight percent is abbreviated as “wt. %”.

As used herein, “active oxygen species” or “reactive oxygen species” are understood as transfer of one or two electrons produces superoxide, an anion with the form O₂″, or peroxide anions, having the formula O₂⁻″ or compounds containing an O—O single bond, for example hydrogen peroxides and lipid peroxides. Such superoxides and peroxides are highly reactive and can cause damage to cellular components including proteins, nucleic acids, and lipids.

As used herein, the term “agent” refers to a therapeutically active compounds or a potentially therapeutic active compound, e.g., an antioxidant. An agent can be a previously known or unknown compound. As used herein, an agent is typically a non-cell based compound, however, an agent can include a biological therapeutic agent, e.g., peptide or nucleic acid therapeutic, e.g., siRNA, shRNA, cytokine, antibody, etc.

As used herein, the terms “amelioration” or “treatment” is understood as meaning to lessen or decrease at least one sign, symptom, indication, or effect of a specific disease or condition. For example, amelioration or treatment of age-related macular degeneration, glaucoma, and/or diabetic retinopathy can be to reduce, delay, or eliminate one or more signs or symptoms of age-related macular degeneration, glaucoma, and/or diabetic retinopathy including, but not limited to, a reduction in night vision, a reduction in overall visual acuity, a reduction in visual field, a reduction in the cone density in one or more quadrants of the retina, thinning of retina, particularly the outer nuclear layer, reduction in a- or b-wave amplitudes on scotopic or photopic electroretinograms (ERGs); or any other clinically acceptable indicators of disease state or progression. Amelioration and treatment can require the administration of more than one dose of an agent, either alone or in conduction with other therapeutic agents and interventions. Amelioration or treatment does not require that the disease or condition be cured.

As used herein, the term “antioxidant” refers to a molecule for slowing or preventing the oxidation of other molecules. Oxidation is a chemical reaction that transfers electrons from a substance to an oxidizing agent. Such reactions can be promoted by or produce superoxide anions or peroxides. Oxidation reactions can produce free radicals, which start chain reaction that damage cells. Antioxidants terminate these chain reactions by removing free radical intermediates and inhibit other oxidation reactions by being oxidized themselves. As a result, antioxidants are often reducing agents such as thiols, ascorbic acid or polyphenols. Antioxidants include, but are not limited to, α-tocopherol, ascorbic acid, Mn(III)tetrakis (4-benzoic acid) porphyrin, α-lipoic acid, and n-acetylcysteine.

As used herein, the term “auxiliary agent” refers to an agent able to reduce at least one side effect of an antioxidant drug. An auxiliary agent comprises a compound able, in the absence of the antioxidant drug, to reduce or prevent a condition associated with at least one side effect of the antioxidant drug.

As used herein, the term “co-administration” refers to the administration of one or more agents to a subject such that the agents are present and active in the subject at the same time. Co-administration does not require a preparation of an admixture of the agents or simultaneous administration of the agents.

As used herein, the terms “effective amount” or “effective doses” refer to that amount

of an agent to product the intended pharmacological, therapeutic or preventive results. The pharmacologically effective amount results in the amelioration of one or more signs or symptoms of a disease or condition or the advancement of a disease or conditions or causes the regression of the disease or condition. For example, a therapeutically effective amount preferably refers to the amount of a therapeutic agent that decreases vision loss, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such as treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as an improvement of symptoms, a cure, a reduction in disease signs or symptoms, extension of life, improvement in quality of life, or other effect generally recognized as positive by medical doctors familiar with treating the particular type of disease or condition.

As used herein, the phrase “oxidative stress related ocular disorders” includes, but is not limited to, age-related macular degeneration, macular degeneration including age-related macular degeneration, glaucoma, and/or diabetic retinopathy, Lebers optic neuropathy, and optic neuritis.

As used herein, the terms “peroxidases” or “a peroxide metabolizing enzyme” refer to a large family of enzymes that typically catalyze a reaction of the form:

ROOR₁+electron donor (2e−)+2H+→>ROH+R₁OH.

For many of these enzymes the optimal substrate is hydrogen peroxide, wherein each R is H, but others are more active with organic hydroperoxides such as lipid peroxides. Peroxidases can contain a heme cofactor in their active sites, or redox-active cysteine or selenocysteine residues.

As used herein, the term phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediaminetetraacetic acid (EDTA), sorbitol, tartaric acid, phosphoric acid, and the like.

Formulations of the present invention include those suitable for oral, nasal, topical, transdermal, buccal, sublingual, intramuscular, intraperotineal, intraocular, intravitreal, posterior juxtascleral, anterior juxtascleral, retrobulbar, subretinal, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, “plurality” is understood to mean more than one. For example, a plurality refers to at least two, three, four, five, or more.

As used herein, the term a “polypeptide” or “peptide” is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full-length proteins (e.g., fully processed proteins) as well as shorter amino acids sequences (e.g., fragments of naturally occurring proteins or synthetic polypeptide fragments).

As used herein, “prevention” is understood as to limit, reduce the rate or degree of onset, or inhibit the development of at least one sign or symptom of a disease or condition particularly in a subject prone to developing the disease or disorder. For example, a subject having a mutation in a gene, such as the opsin gene, is likely to develop age-related macular degeneration, glaucoma, and/or diabetic retinopathy. The age of onset of one or more symptoms of the disease can sometimes be determined by the specific mutation. Prevention can include the delay of onset of one or more signs or symptoms of age-related macular degeneration, glaucoma, and/or diabetic retinopathy and need not be prevention of appearance of at least one sign or symptom of the disease throughout the lifetime of the subject. Prevention can require the administration of more than one does of an agent or therapeutic.

As used herein, the term “small molecule” refers to a compound, typically an organic

compound, having a molecular weight of no more than about 1500 Da, 1000 Da, 750 Da, or 500 Da. In an embodiment, a small molecule does not include a polypeptide or nucleic acid including only natural amino acids and/or nucleotides.

As used herein, the term “subject” refers to living organisms, in particular, humans. In certain embodiments, the living organism is an animal, in certain preferred embodiments, the subject is a mammal, in certain embodiments, the subject is a domesticated mammal or a primate including a non-human primate. Examples of subject include humans, monkeys, dogs, cats, mice, rates, cows, horses, goats, and sheep. A human subject may also be referred to as a subject or patient.

As used herein, a subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition or syndrome. Methods for identification of subjects suffering from or suspected of suffering from conditions such as age-related macular degeneration, glaucoma, and/or diabetic retinopathy is within the ability of those in the art. Subjects suffering from, and suspected of suffering from, a specific disease, condition, or syndrome are not necessarily two distinct groups.

As used herein, the term “superoxide dismutase” refers to an enzyme that dismutation of superoxide into oxygen and hydrogen peroxide. Examples include, but are not limited to SOD1, SOD2, and SOD3. Sod1 and SOD3 are two isoforms of Cu—Zn-containing superoxide dismutase enzymes exists in mammals. Cu—Zn-SOD or SOD1, is found in the intracellular space, and extracellular SOD (ECSOD or SOD3) predominantly is found in the extracellular matrix of most tissues.

As used herein, the phrase “therapeutically effective amount” refers to an amount of an agent which is effective, upon single or multiple does administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone

or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, PA, 1985). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

The present invention is directed to the use of an implant with diNACA to treat age-related macular degeneration, glaucoma, and/or diabetic retinopathy. In one embodiment, the present invention includes a method for the treatment of age-related macular degeneration in a human that comprises administering to the human therapeutically effective amount of diNACA. In some embodiments, the diNACA is provided in or with a pharmaceutically acceptable carrier. In other embodiments, the diNACA implant is administered in or about the eye, e.g., intraocularly, subretinally, intravitreally, posterior juxtascleral, anterior juxtascleral, retrobulbar, intramuscularly, or topically. The implants of the present invention can be injected into one or more non-limiting locations, such as pre-determined locations, that can include, e.g., intravitreal, intrastromal, intracameral, subtenon, retinal, subretinal, retrobulbar, peribulbar, suprachoroidal, subchoroidal, conjunctival, subconjunctival, episcleral, posterior juxtascleral, anterior juxtascleral, circumcorneal, topical, and tear duct.

Administration of a therapeutic agent such as an antioxidant through the use of one or more intraocular implants may improve the treatment of ocular diseases or conditions involving oxidative stress. Implants comprise a pharmaceutically acceptable polymeric composition and are formulated to release one or more pharmaceutically active agents over an extended period of time. In certain embodiments involving the delivery of one agent, the dosage regimen may be formulated to provide one drug to the anterior or posterior segment of the eye. For example, for ocular drug delivery to the posterior region of the eye, the implant would likely be introduced as an intravitreal implant, inserted into the vitreous cavity.

In certain embodiments involving the delivery of more than one agent, the dosage regiment may be formulated to provide two or more drugs to the posterior segment of the eye under different dosage regimens. For example, the dosage of the antioxidant in an implant may be made to be discontinuous over the treatment period while a non-discontinuous dosage of an auxiliary agent is administered in an implant over the same overall time period. The implant containing the antioxidant and the implant containing the auxiliary agent may be different implants or the same implant comprising means of differentially administering the antioxidant and auxiliary agent, such means including different coatings or shells which may contain, neither, one or both drugs, or covalent linkage of one or both drugs to a biodegradable polymer of the implant by way of a biodegradable linkage, thus permitting regulation of the delivery of one or more drug over the time of the treatment. The implants are effective to provide a therapeutically effective dosage of the agent or agents directly to a region of the eye to treat one or more ocular diseases or conditions involving oxidative stress. Thus, with a single administration, therapeutic agents will be made available at the site where they are needed and may be maintained for an extended period of time, rather than subjecting the patient to repeated injections or, in the case of self-administered drops, ineffective treatment with only limited bursts of exposure to the active agent or agents.

As used herein, the term “auxiliary agents” refers to agents such as lubricants, preservatives, stabilizers, wetting agents, emulsifiers, salts for influencing osmotic pressure, buffers, colorings, flavorings and/or aromatic substances, and the like, which do not deleteriously interact with the antioxidants of the formulation.

One such intraocular implant in accordance with the disclosure herein comprises a therapeutic component and a drug release sustaining component associated with the therapeutic component. In accordance with the present invention, the therapeutic component comprises, consists essentially of, or consists of, the antioxidant, diNACA. The drug release sustaining component is associated with the therapeutic component to sustain release of a therapeutically effective amount of the antioxidant into an eye in which the implant is placed. The therapeutic amount of the antioxidant is released into the eye for a period of time greater than about two months after the implant is placed in the eye.

For the purposes of this description, we use the following terms as defined in this section, unless the context of the word indicates a different meaning.

As used herein, an “intraocular implant” refers to a device or element or drug product that is structured, sized, or otherwise configured to be placed “in an eye”, including the subconjunctival space. Intraocular implants are generally biocompatible with physiological conditions of an eye and do not cause adverse side effects. Intraocular implants may be placed in an eye without disrupting vision of the eye.

As used herein, a “therapeutic component” refers to a portion of an intraocular implant comprising one or more therapeutic agents or substances used to treat a medical condition of the eye. The therapeutic component may be a discrete region of an intraocular implant, or it may be homogenously distributed throughout the implant. The therapeutic agents of the therapeutic component are typically ophthalmically acceptable, and are provided in a form that does not cause adverse reactions when the implant is placed in an eye.

As used herein, a “drug release sustaining component” refers to a portion of the intraocular implant that is effective to provide a sustained release or extended-release of the therapeutic agents of the implant. A drug release sustaining component may be a biodegradable polymer matrix, or it may be a coating covering a core region of the implant that comprises a therapeutic component.

As used herein, “associated with” means mixed with, dispersed within, coupled to, covering, or surrounding. With respect to intraocular implants which comprise a therapeutic component associated with a biodegradable polymer matrix, “associated with” specifically excludes biodegradable polymeric coatings that may be provided on or around the matrix.

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eye in an ocular region include the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

As used herein, an “ophthalmic or ocular disease” or “ophthalmic or ocular condition” is a disease, ailment or condition which affects or involves the eye or one of the parts or regions of the eye. Broadly speaking the eye includes the eyeball, or globe, the tissues and fluids which constitute the eye, the periocular muscles (such as the oblique and rectus muscles) and the portion of the optic nerve which is within or adjacent to the eye.

An anterior ocular condition is a disease, ailment or condition which affects or which involves an anterior (i.e., front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the retina but in front of the posterior wall of the lens capsule), the lens or the lens capsule and blood vessels and nerve which vascularize or innervate an anterior ocular region or site.

An anterior ocular condition can include a disease, ailment, or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e., reduce intraocular pressure).

A posterior ocular condition is a disease, ailment or condition which primarily affects

or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition, such as for example, acute macular neuroretinopathy; Behet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e., neuroprotection).

As used herein, the term “biodegradable” refers to a material that will breakdown to soluble species or that will degrade under physiologic conditions to smaller units or chemical species that are, themselves, non-toxic (biocompatible) to the subject and capable of being metabolized, eliminated, or excreted by the subject. The terms biodegradable, bioabsorbable, and bioerodible as used herein are equivalent and are used interchangeably herein.

As used herein, the term “nonbiodegradable” refers refers to a material that will not breakdown to soluble species or that will not degrade under physiologic conditions to smaller units or chemical species that are, themselves, non-toxic (biocompatible) to the subject and capable of being metabolized, eliminated, or excreted by the subject. The terms nonbiodegradable, nonbioerodible and nonbioabsorbable as used herein are equivalent and are used interchangeably herein.

As used herein, the term “biodegradable polymer” refers to a polymer or polymers which degrade in vivo, and wherein erosion and/or breakdown of the polymer or polymers over time occurs concurrently with or subsequent to release of the therapeutic agent. Specifically, hydrogels such as methylcellulose which act to release drug through polymer swelling are specifically excluded from the term “biodegradable polymer”. A biodegradable polymer may be a homopolymer, a copolymer, or a polymer comprising more than two different polymeric units.

As used herein, the term “treat”, “treating”, or “treatment” refers to reduction or resolution or prevention of an ocular condition, ocular injury or damage, or to promote healing of injured or damaged ocular tissue.

As used herein, the term “therapeutically effective amount” refers to the level or amount of agent needed to treat an ocular condition, or reduce or prevent ocular injury or damage without causing significant negative or adverse side effects to the eye or a region of the eye.

As used herein, “susceptible to” or “prone to” or “predisposed to” a specific disease or condition or the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200% or more.

As used herein, the term “lactide” refers to lactide and lactic acid monomers.

As used herein, the term “glycolide” refers to glycolide and glycolic acid monomers.

The present invention provides new drug delivery formulations or preparations, and methods of using such formulations and preparations, for extended or sustained drug release into an eye, for example, to achieve sustained desired therapeutic effects. The drug delivery formulations or preparations are in the form of implants or implant elements that may be placed in an eye.

Intraocular implants in accordance with the disclosure herein comprise an antioxidant drug. The antioxidant drug may be present in or on the same implant or different implants. The antioxidant drug may reduce oxidative stress in the eye in an acceptable range.

Such intraocular implants may comprise a therapeutic component and a drug release sustaining component associated with the therapeutic component. In accordance with the present invention, the therapeutic component comprises, consists essentially of, or consists of, an antioxidant drug. The drug release sustaining component is associated with the therapeutic component to sustain release of a therapeutically effective amount of the steroid into an eye in which the implant is placed. The therapeutically effective amount of the antioxidant is preferably released into the eye for a period of time greater than about one or two or more months after the implant is placed in the eye.

In one embodiment, the intraocular implants comprise an antioxidant drug and a biodegradable polymer matrix. The antioxidant drug is associated with a biodegradable polymer matrix that releases drug, such as by dissolving, breaking apart or degrading, at a rate effective to sustain release of a therapeutically effective amount of the antioxidant drug from the implant for a time greater or longer than about one or two or more months from a time the implant is placed in an ocular site or region of an eye. The intraocular implant is biodegradable or bioerodible and provides a sustained release of the antioxidant drug in an eye for extended periods of time, such as for more than a few days, weeks, two months, or for about three months or more and up to about six months or more.

The biodegradable polymer component of the implant may be a mixture of biodegradable polymers, wherein at least one of the biodegradable polymers is a polylactide or poly(lactide-co-glycolide) polymer having a molecular weight less than 40 kiloDaltons (kD). Additionally or alternatively, the implants may comprise a first biodegradable polymer having terminal free acid groups, and a different second biodegradable polymer having terminal free acid groups. Additionally or alternatively, the implants may comprise a first biodegradable polymer having a terminal ester, and a different second biodegradable polymer having terminal ester groups Furthermore, the foregoing implants may comprise a mixture of different biodegradable polymers, each biodegradable polymer having an inherent viscosity in a range of about 0.16 deciliters/gram (dL/g) to about 0.24 dL/g. Examples of suitable biodegradable polymers include polymers synthesized from lactide monomer, glycolide monomer and mixtures thereof. Other examples of suitable biodegrdable polymers include polymers synthesized from lactic acid monomer, glycolic acid monomer and mixtures thereof.

In another embodiment, intraocular implants comprise a therapeutic component that comprises an antioxidant drug, and a polymeric outer layer covering the therapeutic component. The polymeric outer layer may include one or more orifices or openings or holes that are effective to allow a liquid to pass into the implant, and to allow the steroid to pass out of the implant. The therapeutic component is provided in a core or interior portion of the implant, and the polymeric outer layer covers or coats the core. The polymeric outer layer may include one or more biodegradable portions. The implant can provide an extended release of the steroid for more or longer than about two months, and for more than about one year, and even for more than about five or about ten years.

In one embodiment, the polymeric outer layer of the implant may comprise two or more layers or coats of biodegradable material, with each such layer having a different composition or rate of degradation than the layer immediately adjoining it. For example, the polymeric outer layer of the implant may comprise concentric rings or nested coatings comprising a first layer, wherein the first layer may comprise, for example, a biodegradable polymer and the absence of antioxidant drug, a biodegradable polymer comprising a therapeutically effective amount of an antioxidant drug, a biodegradable polymer comprising an amount of an auxiliary agent effective to reduce at least one side effect of antioxidant drug, and a biodegradable polymer comprising a therapeutically effective amount of antioxidant drug and an amount of an auxiliary agent effective to reduce at least one side effect of a steroid, and a biodegradable polymer without any added drug.

A second layer may also comprise, for example, a biodegradable polymer and the absence of antioxidant drug, a biodegradable polymer comprising a therapeutically effective amount of an antioxidant drug, a biodegradable polymer comprising an amount of an auxiliary agent able to reduce at least one side effect of an antioxidant drug, and a biodegradable polymer comprising a therapeutically effective amount of a steroid and an amount of an auxiliary agent able to reduce at least one side effect of a steroid, and a biodegradable polymer without any added drug, with the additional provisos that the first and second layers are located adjoining one another in the biodegradable implant, that the first and second layers are not identical, and that the first layer is designed to erode substantially before the second layer.

Additional layers may be present; preferably, each such layer will not be identical to the layers immediately surrounding it.

It is well known that long-term ophthalmic treatment with drugs must be monitored closely due to potential toxicity and long-term side effects. For example, adverse reactions listed for conventional ophthalmic dexamethasone preparations include: glaucoma (with optic nerve damage, visual acuity and field defects, and ocular hypertension), posterior subcapsular cataract formation, and secondary ocular infection from pathogens including herpes simplex. Additional hazardous side-effects upon conventional topical treatment with steroids may comprise hypertension, hyperglycemia, increased susceptibility to infection, and peptic ulcers.

Age-related macular degeneration (“AMD”) comprises a large group of inherited vision disorders that cause progressive loss of photoreceptor cells of the retina, leading to severe vision impairment and often incurable blindness. The most common form of AMD is a rod-cone dystrophy, in which the first symptom is night blindness, followed by progressive loss in the peripheral visual field in daylight, and eventually leading to blindness after several decades. As a common pathology, rod photoreceptors die early, whereas light-insensitive, morphologically altered cone photoreceptors persist longer.

Diabetic retinopathy (DR), sometimes referred to as diabetic eye disease, in which diabetes mellitus leads to damage to the retina, and is a leading cause of blindness. Typically, DR affects up to 80 percent of diabetic patients. Importantly, the longer a patient has diabetes, the higher the chances of developing diabetic retinopathy. In the United States, diabetic retinopathy accounts for 12% of all new cases of blindness, and is the leading cause of blindness in patients aged 20 to 64.

Glaucoma described several eye diseases that result from damage to the optic nerve leading to loss of vision. Typical symptoms of glaucoma include, e.g., eye pain, blurred vision, mid-dilated pupil, redness of the eye, and nausea. An increase in intraocular pressure is a major risk factor for glaucoma, as are a family history of glaucoma and high blood pressure, however, the etiology of glaucoma is still under investigation.

(2R,2R′)-3,3′-disulfanediyl bis(2-acetamidopropanamide) (diNACA) also referred to

herein as NPI-002, has the chemical structure:

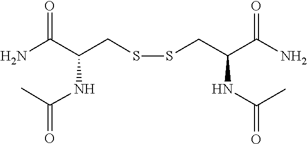 [SMILES: CC(=O)N[C@@H](CSSC[C@H](NC(C)=O)C(N)=O)C(N)=O]

(2R,2R′)-3,3′-disulfanediyl bis(2-acetamidopropanamide) (diNACA), the dimer form of N-acetyl-L-cysteineamide, can be used in the treatment of ophthalmic disease and conditions involving oxidatve stress, including but not limited to, age-related macular degeneration, cataract, corneal endothelial cell loss or protection, glaucoma, diabetic retinopathy, presbyopia, and retinitis pigmentosa.

Gluthathione (GSH) is a tripeptide, c-L-glutamyl-L-cysteinyl-glycine, found in all mammalian tissues. It has several important functions including detoxification of electrophiles, scavenging ROS<maintaining the thiol status of proteins, and regeneration of the reduced forms of vitamins C and E. GSH is the dominant non-protein thiol in mammalian cells; as such it is essential in maintaining the intracellular redox balance and the essential thiol status of proteins. Also, it is necessary for the function of some antioxidant enzymes such as the glutathione peroxidases.

Intracellular GSH levels are determined by the balance between production and loss. Production results from de novo synthesis and regeneration of GSH from GSSG by GSSG reductase. Generally there is sufficient capacity in the GSSG reductase system to maintain all intracellular GSH in the reduced state, so little can be gained by ramping up that pathway. The major source of loss of intracellular GSH is transport out of cells. Intracellular GSH levels range from 1-8 mM while extracellular levels are only a few μM; this large concentration gradient essentially precludes transport of GSH into cells and once it is transported out of cells, it is rapidly degraded by γ-glutamyltranspeptidase. Inhibition of GSH transporters could theoretically increase intracellular GSH levels, but is potentially problematic because the transporters are not specific for GSH and their suppression could lead imbalance of other amino acids and peptides. Thus, intracellular GSH levels are modulated primarily by changes in synthesis.

GSH is synthesized in the cytosol of virtually all cells by two ATP-requiring enzymatic steps: L-glutamate+L-cysteine+ATP [→]γ-glutamyl-L-cysteine+ADP+Pi and γ-glutamyl-L-cysteine+L-glycine+ATP [→]GSH+ADP+Pi. The first reaction is rate-limiting and is catalyzed by glutamate cysteine ligase (GCL, EC 6.3.2.2). GCL is composed of a 73 Kd heavy catalytic subunit (GCLC) and a 30 Kd modifier subunit (GCLM), which are encoded by different genes. GCCL is regulated by nonallosteric competitive inhibition of GSH (Ki=2.3 mM) and by the availability of L-cysteine. The apparent Km of GLC for glutamate is 1.8 mM and intracellular glutamate concentration is roughly 10-fold higher so that glutamate is not limiting, but the Km for cysteine is 0.1-0.3 mM, which approximates its intracellular concentration. The second reaction is catalyzed by GSH synthase (GS, EC 6.3.2.3), which is 118 Kd and composed of two identical subunits. While GS is not felt to be important in regulation of GSH synthesis under normal conditions, it may play a role under stressful conditions because in response to surgical trauma, GSH levels and GS activity were reduced while GCL activity was unchanged. Furthermore, compared to increased expression of GCLC alone, increased expression of both GCLC and GS resulted in higher levels of GSH. In order to maximize the effects of increasing synthetic enzymes, it is necessary to provide increased levels of cysteine. In cultured neurons, 90% of cysteine uptake occurs through by the sodium-dependent excitatory amino acid transporter (EAAT) system. There are five EAATs and cysteine uptake by neurons occurs predominantly by EAAT3 more commonly known as excitatory amino acid carrier-1 (EAAC1). Under normal circumstances most EAAC1 is in the ER and only translocates to the plasma membrane when activated. This translocation is negatively regulated by glutamate transporter associated protein 3-18 (GTRAP3-18) and suppression of GTRAP3-18) increased GSH levels in neurons. Thus, internalization of cysteine provides a road block for GSH synthesis, but fortunately it can be bypassed by N-acetylcysteine (NAC) which readily enters cells even in the absence of activated EAAC1. Systemically administered NAC gains access to the CNS, increases GSH levels, and provides benefit in neurodegenerative disorders in which oxidative stress is an important part of the pathogenesis.

All cellular compartments must be protected against oxidative damage, including the cytoplasm, mitochondria and the nucleus. The present inventors have previously performed gene transfer of enzymes that detoxify reactive oxygen species, but that approach requires expression of two enzymes in the cytoplasm and two enzymes in mitochondria. In contrast, the present invention provides for protection of all cellular compartments with expression of only two enzymes in the cytosol because GSH is able to diffuse everywhere throughout cells.

NAC is used for the treatment of acetaminophen overdose at a dose of 140 mg/kg as the loading dose, followed by 70 mg/kg every 4 hours for 17 doses, starting 4 hours after the loading dose. In clinical studies, NAC has been administered orally from 400 to 1000 mg once daily and from 200 to 600 mg three times daily. However, following an oral dose of 600 mg in humans, NAC is rapidly absorbed and then rapidly cleared. The plasma half-life of NAC has been reported to be 2.5 hours and no NAC is detectable 10-12 hours after administration. During absorption, NAC is rapidly metabolized to cysteine, which is a direct precursor of glutathione. Based on this evidence, including that NACA is a precursor and/or carrier for NAC, it was expected that NACA would act similarly to NAC in vivo. However, the present inventors demonstrate that NACA and diNACA act very differently from NAC for the treatment of age-related macular degeneration, glaucoma, and/or diabetic retinopathy.

In accordance with an embodiment, the present invention provides a method for the prevention, amelioration, or treatment of a disease or condition associated with oxidative stress in a subject comprising administration of a therapeutically effective amount of NACA, to increase the amount of glutathione expressed in the tissues of the subject.

Intraocular implants have been developed which can release drug loads over various time periods. These implants, which when inserted into an eye, such as, without limitation, the vitreous of an eye or the subconjunctival space, provide therapeutic levels of a steroid and/or auxiliary agent for extended periods of time (e.g., for about 2 months or more). The implants disclosed are effective in treating ocular conditions, such as posterior ocular conditions.

In one embodiment of the present invention, an intraocular implant comprises a biodegradable polymer matrix. The biodegradable polymer matrix is one type of a drug release sustaining component. The biodegradable polymer matrix is effective in forming a biodegradable intraocular implant. The biodegradable intraocular implant may comprise a steroid and or auxiliary agent associated with the biodegradable polymer matrix. Such association may be “passive”, such as through co-extrusion of the active agent(s) with the biodegradable polymer, or “active”, by being joined, or coupled to the polymer through covalent chemical bonds, chelation, strong hydrogen bonding, ionic interaction, and the like. The matrix degrades at a rate effective to sustain release of a therapeutically effective amount of the steroid for a time greater than about two months from the time in which the implant is placed in ocular region or ocular site, such as the vitreous of an eye.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the forgoing guidelines.

Ranges provided herein are understood to be shorthand for all of the values within the range.

As used herein, the embodiments of this invention are defined to include pharmaceutically acceptable derivatives thereof. A “pharmaceutically acceptable derivative” refers to any pharmaceutically salt, ester, salt of an ester, or other derivative of a compound of this invention which, upon administration to a recipient, is capable of providing (directly or indirectly) a compound of this invention. Particularly favored derivatives are those that increase the bioavailability of the compounds of this invention when such compounds are administered to a mammal (e.g., by allowing an orally administered compound to be more readily absorbed into the blood, to increase serum stability or decrease clearance rate of the compound) or which enhance delivery of the parent compound to a biological compartment (e.g., the brain or lymphatic system) relative to the parent species. Derivatives include derivatives where a group which enhances aqueous solubility or active transport through the gut membrane is appended to the structure of formulae described herein.

The embodiments of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion. Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-napthalenesulfonate, nicotinate, nitrate, palmoate, phosphate, picrate, pivalate, propionate, salicylate, succinate, sulfate, tartrate, tosylate, and undeconaoate. Salts derived from appropriate bases include alkali metal (e.g., sodium), alkaline earth metal (e.g., magnesium), ammonium and N-(alkyl)4+ salts. This invention also envisions the quaternization of any basic nitrogen-containing groups of the compounds disclosed herein. Water or oil-soluble or dispersible products may be obtained by such quaternization.

The embodiments of the invention can, for example, be administered by injection, intraocularly, intravitreally, retrobulbar, posterior juxtascleral, anterior juxtascleral, subretinal, intravenously, intraarterially, subdermally, intramuscularly, or subcutaneously; or orally, buccally, nasally, transmucosally, directly to a diseased organ by catheter, topically, or in an ophthalmic preparation, with a dosage ranging from about 0.001, 0.01, 0.1, 0.5, 1, 2, 2.5, 3, 4, 6, 7, 7.5, 8, 9, 10, 15, 20, 25, 30, 40, 50, 60, 70, 75, 80, 90 to about 100 mg/kg of body weight, or according to the requirements of the particular drug and more preferably from 0.5-10 mg/kg of body weight. It is understood that when a compound is delivered directly to the eye, considerations such as body weight have less bearing on the dose.

Frequency of dosing will depend on the agent administered, the progression of the disease or condition in the subject, and other considerations known to those of skill in the art. For example, pharmacokinetic and pharmacodynamics considerations for compositions delivered to the eye, or even compartments within the eye, are different, e.g., clearance in the subretinal space is very low. Therefore, dosing can be as infrequent as once a month, once every three months, once every six months, once a year, once every five years, or less. If systemic administration of antioxidants is to be performed in conjunction with administration of expression constructs to the subretinal space, it is expected that the dosing frequency of the antioxidant will be higher than the expression construct, e.g., one or more times daily, one or more times weekly.

Dosing may be determined in conjunction with monitoring of one or more signs or symptoms of the disease, e.g., visual acuity, visual field, night visions, etc. The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity ad course of the disease, condition or symptoms, the patient's disposition to the disease, condition or symptoms and the judgment of the treating physician.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEEN® or SPAN® and/or other similar emulsifying agents or bioavailability enhancers which are commonly used in the manufacture of pharmaceutically acceptable solid, liquid, or other dosage forms may also be used for the purposes of formulation.

In one or more embodiments, diNACA is administered in daily doses of about 0.5 to 150 mg/Kg. In other embodiments, diNACA is administered two or three times daily. In another aspect, diNACA is administered with a second active agent selected from ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediaminetetraacetic acid (EDTA), sorbitol, tartaric acid, phosphoric acid, and the like.

In some embodiments, the dose of diNACA for administration is, 100, 150, 150, 300, 333, 400, 500, 600, 700, 750, 800, 900, 1,000, 2,500, 5,000, 7,500, or 10,000 mg per implanted dose. In another aspect, the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per implanted dose. In another aspect, the diNACA implant is administered prophylactically to prevent the diseases of the eye.

In another embodiment, the present invention includes a method for the treatment of age-related macular degeneration, glaucoma, and/or diabetic retinopathy comprising: identifying a human in need of treatment for age-related macular degeneration; and administering to the human a therapeutically effective amount of diNACA sufficient to treat age-related macular degeneration, glaucoma, and/or diabetic retinopathy. It will be understood that, as with the other embodiments defined above, diNACA is administered in daily doses of about 0.5 to 150 mg/Kg. In another aspect, diNACA is administered two or three times daily. In another aspect, NACA is administered with a second active agent as disclosed above.

In another aspect, an additional dose of diNACA for administration is 100, 150, 150, 300, 333, 400, 500, 600, 700, 750, 800, 900, 1,000, 2,000, 2,500, 3,000, 4,000, 5,000, 6,000, 7,000, 7,500, 8,000, 9,000, or 10,000 mg per dose. In another aspect, the dose for administration is 0.1-0.25, 0.1-0.4, 0.35-0.5, 0.5-1, 1-2, 1-3, 1-4, 1-5, 1-2.5, 2.5-3.5, 4-6, 5-8, 6-9, 7-10 grams per dose. In another aspect, the NACA is delivered orally via a mini-tablet, capsule, tablet, effervescent, dual release, mixed release, sachet, powder, or liquid. In another aspect, diNACA is administered prophylactically to prevent age-related macular degeneration, glaucoma, and/or diabetic retinopathy.

The diNACA formulations described herein may also be delivered to the eye via ocular topical, intravitreal injection, posterior juxtascleral injection, anterior juxtascleral injection, and periocular injection routes. In one embodiment of the present invention, the amount of active agent, or poorly water-soluble agent, or biologic will be from about 0.001% to 30% weight to volume of the active agent in a solution for intravitreal administration. In other embodiments, the amount of active can be from 0.05% to 20% weight to volume and in some cases from 0.1% to 18% weight to volume. It is contemplated that any active agent that is poorly water soluble, or slightly soluble, may be included in the compositions of the present invention. In other instances, highly water-soluble active or inactive agents may also be included in the compositions of the present invention.

For example, the present invention may be delivered is a thick or viscous vehicle that allows for extended release of the active agent. For example, the diNACA can be provided in any poly(ethylene glycol) (PEG) with a molecular weight greater than 500, 1000, 1,5000, or 2000 in the compositions and methods of the invention. PEGs for use in the compositions and methods of the invention can also include PEG 3000, PEG 4000, PEG 6000, PEG 8000, PEG 20000, or higher molecular PEGs may be utilized in the compositions and methods of the invention.

The diNACA formulations of the present invention provide a number of advantages over conventional formulations because PEGs can successfully solubilize poorly soluble compounds, allowing the preparation of an efficacious ophthalmologically acceptable intravitreal, posterior juxtascleral (PJ), anterior juxtascleral, (AJ), and/or periocular formulation for local ocular delivery. Bioavailability of the drug can be modulated by controlling the molecular weight, or mixture of molecular weights, of the PEG used in the formulation. Furthermore, the preparation can be injected using a 27- or 30-gauge needle. Toxicity of the active agent can also be reduced or suitably modulated by extending its release. Another advantage of the present invention is that the stability of biologics is improved in the PEG-based solid dosage form described herein.

The formulation of the invention may further comprise a lipid to modulate the delivery of the drug and to extend the duration. Some examples of a lipid include triglycerides, diglycerides, monoglycerides, propylene glycol esters, PEF esters of fatty acid and their mixtures. Preferred lipids include glyceryl monolaurate; glyceryl dilaurate; glyceryl monomyristate; glyceryl dimyristate; glyceryl monopalmitate; glyceryl dipalmitate; glyceryl monostearate; glyceryl distearate; glyceryl monooleate; glyceryl dioleate; glyceryl monolinoleate; glyceryl dilinoleate; glyceryl monoarachidate; glyceryl diarachidate; glyceryl monobehenate; glyceryl dibehenate; diethylene glycol monostearate; propylene glycol monostearate; glyceryl monostearate; glyceryl monolinoleate; glyceryl monooleate; glyceryl monopalmitate; and mixtures thereof. A preferred example of the lipid is glyceryl palmitostearate. The concentration of a lipid is generally less than 31 weight percent (wt %), but often less than 14 wt % and in some cases less than 8 wt %.

The specific dose level of the active agent for any particular human or animal depends upon a variety of factors, including the activity of the active compound used, the age, body weight, general health, time of administration, route of administration, and the severity of the pathologic condition undergoing therapy.

The present invention generally relates to devices and methods to treat an eye of a patient, and more specifically to intraocular implants that provide extended release of a therapeutic agent to an eye in which the implant is placed. After elution and exhaustion of the active agent from the implant, the therapeutic effect is no longer rendered.

To remedy, the use of sustained-released drug delivery systems may be employed. In 2000, Jaffe et al. reported using compressed fluocinolone acetonide pellets coated with silicone and poly(vinyl alcohol) as a fluocinolone sustained delivery device (Jaffe, et al., 2000). Jaffe et al. (2000) obtained release rates of 1.9±0.25 4/day (6 months) and 2.2±0.6 μg/day (45 days) for the 2-mg device and 15-mg device, respectively. The duration of release for the 2-mg and 15-mg device was estimated to be 2.7 and 18.6 years, respectively. U.S. Pat. Nos. 6,217,895 and 6,548,078 disclose sustained release implants for delivering a corticosteroid, such as fluocinolone acetonide, to an eye. However, fluocinolone acetonide intravitreal implants made by Control Delivery Systems (the assignee of U.S. Pat. Nos. 6,217,895 and 6,548,078) were only partially successful and led to the development of cataracts and increased intraocular pressure.

Additional biocompatible implants for placement in the eye have been disclosed in a number of patents, such as U.S. Pat. Nos. 4,521,210; 4,853,224; 4,997,652; 5,164,188; 5,501,856; 5,766,242; 5,824,072; 5,869,079; 6,074,661; 6,331,313; 6,369,116; 6,699,493, and 6,726,918.

Other intravitreal therapeutic approaches are described in U.S. application Ser. No. filed Oct. 14, 2004; Ser. No. 11/039,192, filed Jan. 19, 2005; and 60/587,092, filed Jul. 12, 2004.

It would be advantageous to provide eye implantable drug delivery systems, such as intraocular implants, and methods of using such systems, that are capable of releasing a therapeutic agent at a sustained or controlled rate for extended periods of time and in amounts with few or no negative side effects.

The biodegradable polymer may comprise a plurality of hydroxyl groups to which said agent may be joined by a biodegradable linkage. Biodegradable or biocleavable linkages are defined as types of specific chemical moieties or groups that can be used within the chemical substances that covalently reversibly couple or crosslink a therapeutic agent and/or an auxiliary agent to a biodegradable polymer comprised in the implant. Thus, such linkages may be contained in certain embodiments of the instant invention that provide the function of controlled release of a steroid and/or auxiliary agent. In certain embodiments of the present invention an implant system comprising one or more implant is structured such that the therapeutic agent and the auxiliary agent are released at different rates or different times following implantation of the implant(s). Biocleavable linkages or bonds can be distinguishable by their structure and function and non-limiting examples are provided here under distinct categories or types.

One such category comprises the disulfide linkages that are well known for covalent coupling. Such linkages are stable under oxidizing conditions but can be cleaved under reducing conditions. For drug delivery, they may be more useful for shorter periods in vivo since they are cleaved relatively easily. A simple ester bond is another preferred type that may easily be formed between an acid and an alcohol. Another preferred type is any imidoester formed from alkyl imidates. Also included are maleimide bonds as with sulfhydryls or amines used to incorporate a biocleavable linkage.

Another category in this invention comprises acid-cleavable biocleavable linkages. The preferred biocleavable linkages for such release of active agents and other moieties. One such type is an acid-sensitive (or acid-labile) hydrazone linkage as described by Greenfield, et al. (1990), and references therein.

Other types of acid-labile linkages include polyortho or diortho ester linkage; examples of such linkages are disclosed in J. Heller, et al. (1985), Heller et al., (1993) and Ahmad, et al. (1979), and references therein. Also, useful may be acid labile phosphonamide linkages disclosed by Rahil, et al. (1981) and Jeong, et al. (2003).

Implants may be placed in an ocular region to treat a variety of ocular conditions, including conditions that affect an anterior region or posterior region of an eye. For example, the implants may be used to treat many conditions of the eye, including, without limitation, maculopathies and retinal degeneration, uveitis, retinitis, choroiditis, vascular diseases, and exudative diseases, proliferative disorders, infectious disorders, genetic disorders, tumors, trauma, and surgery, retinal tears or holes, or similar. In particular, treatment of retinal conditions are particularly advantageous by means if insertion, injection or other intravitreal delivery, or subconjunctival delivery of such implants.

Kits in accordance with the present invention may comprise one or more of the present implants, and instructions for using the implants. For example, the instructions may explain how to administer the implants to a patient, and types of conditions that may be treated with the implants.

Kits may also include an injector device used to deliver the implant to the eye. Kits, implants and injectors are sterile.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

Additional aspects and advantages of the present invention are set forth in the following description and claims, particularly when considered in conjunction with the accompanying drawings and examples.

The antioxidant of the implant is preferably from about 10 to 90% by weight of the implant. More preferably, the antioxidant about 50 to about 80% by weight of the implant or 10% to 49% of the implant or 20% to 60% of the implant. In a preferred embodiment, the antioxidant comprises about 30% by weight of the implant. In a preferred embodiment, the antioxidant comprises about 35% by weight of the implant. In a preferred embodiment, the antioxidant comprises about 40% by weight of the implant. In a preferred embodiment, the antioxidant comprises about 45% by weight of the implant. In a preferred embodiment, the antioxidant comprises about 50% by weight of the implant. In another embodiment, the antioxidant comprises about 70% by weight of the implant.

Suitable polymeric materials or compositions for use in the implant include those materials which are compatible, that is biocompatible, with the eye so as to cause no substantial interference with the functioning or physiology of the eye. Such materials preferably are at least partially and more preferably substantially completely biodegradable, bioabsorbable or bioerodible.

Examples of useful polymeric materials include, without limitation, such materials derived from and/or including organic esters and organic ethers, which when degraded result in physiologically acceptable degradation products, including the monomers. Examples of useful organic ester polymeric materials include, without limitation, polylactide, polyglycolide, polycaprolactone, poly(lactide-co-glycolide) poly(lactide-co-caprolactone), poly(lactic acid), poly(glycolic acid), poly(lactic acid-co-glycolic acid), polyhydroxbutyrate and the like. Also, polymeric materials derived from and/or including, anhydrides, amides, orthoesters and the like, by themselves or in combination with other monomers, may also find use. The polymeric materials may be ring-opening or addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g., non-oxo-carbonyl, such as carboxylic acid ester, and the like. The nitrogen may be present as amide, cyano and amino. The polymers set forth in Heller (1987), which describes encapsulation for controlled drug delivery, may find use in the present implants.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, D-lactide, L-lactide, racemic lactide, glycolide, polycaprolactone, and combinations thereof. Herein the term lactide pertains to lactide and lactic acid. Herein the term glycolide pertains to glycolide and glycolic acid. Generally, by employing the L-lactate or D-lactate, a slowly biodegrading and/or bioabsorbing polymer or polymeric material is achieved, while biodegradation and/or bioabsorption is substantially enhanced with the lactate racemate.

Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, poly(vinyl alcohol), polyesters, polyethers and combinations thereof which are biocompatible and may be biodegradable and/or bioabsorbable and/or bioerodible.

Some preferred characteristics of the polymers or polymeric materials for use in the present invention may include biocompatibility, compatibility with the therapeutic component, ease of use of the polymer in making the drug delivery formulations or preparations of the present invention, a half-life in the physiological environment of at least about 5 hours, preferably greater than about one day, not significantly increasing the viscosity of the vitreous, and water insolubility.

The biodegradable polymeric materials which are included to form the matrix are desirably subject to enzymatic or hydrolytic instability. Water-soluble polymers may be cross-linked with hydrolytic or biodegradable unstable cross-links to provide useful water-insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended-release profile of the implant is the relative average molecular weight of the polymeric composition employed in the implant. Different molecular weights of the same or different polymeric compositions may be included in the implant to modulate the agent release profile. In certain implants, the relative average molecular weight of the polymer will range from about 10 to about 70 kD, usually from about 10 to about 55 kD, more usually from about to about 45 kD, and most usually less than about 40 kD.

In some implants, copolymers made from glycolide and lactide monomers are used, where the rate of biodegradation is controlled by the ratio of glycolide monomer to lactide monomer incorporated into the polymer chain. The most rapidly degraded copolymer has roughly equal amounts of glycolide and lactide incorporation. Homopolymers, or copolymers having ratios other than equal, take longer to resorb. The ratio of glycolide to lactide incorporated will also affect the brittleness of the implant, where a more flexible implant is desirable for larger geometries. The percent of lactide monomer incorporated in the poly(lactide-co-glycolide) (PLG) copolymer can be 0-100%, preferably about 15-85%, more preferably about 35-65%. In some implants, a 50/50 PLG copolymer is used.

The biodegradable polymer matrix of the intraocular implant may comprise a mixture of two or more biodegradable polymers. For example, the implant may comprise a mixture of a first biodegradable polymer and a different second biodegradable polymer. One or more of the biodegradable polymers may have terminal acid groups. One or more of the biodegradable polymers may have ester end groups. In certain implants, the matrix comprises a first biodegradable polymer having terminal acid groups, and a different second biodegradable polymer having terminal acid groups. The first biodegradable polymer may be a poly(DL-lactide-co-glycolide). The second biodegradable polymer may be a poly(DL-lactide).

Release of a drug from an bioabsorbable polymer is the consequence of several mechanisms or combinations of mechanisms. Some of these mechanisms include desorption from the implants surface, dissolution, diffusion through porous channels of the hydrated polymer and erosion. Erosion can be bulk or surface or a combination of both. As discussed herein, the matrix of the intraocular implant may release drug at a rate effective to sustain release of a therapeutically effective amount of the steroid for more than three months after implantation into an eye. In certain implants, therapeutic amounts of the drug are released for more than four months after implantation. For example, an implant may comprise an antioxidant, and the matrix of the implant degrades at a rate effective to sustain release of a therapeutically effective amount of antioxidant for about three months after being placed in an eye. As another example, the implant may comprise antioxidant, and the matrix releases drug at a rate effective to sustain release of a therapeutically effective amount of antioxidant for more than three months, such as from about three months to about six months.

The rate of release of a drug from an implant of the present invention may be related to the physical structure of the implant. In a simple but very useful embodiment, the biodegradable polymer of the present invention may comprise a substantially homogeneous matrix mixed with the active agent(s). Upon drying and formulation of the matrix into an intraocular implant, the active agent may be substantially homogeneously distributed in the matrix. In such an embodiment, the release characteristics of the agent is largely determined by the nature of the solvent and the rate of degradation of the matrix.

In another embodiment the implant may comprise layers or shells of differently formulated biodegradable polymer with which the therapeutic agent and/or auxiliary agent may be associated and released. Concentrations of polymer, therapeutic agent and/or auxiliary agent may differ between layers, or the biodegradable polymer may be formulated to deliver such agents at different rates, according to different release profiles, or over different time periods that other layers, or than a core portion of the implant. Each layer may be formulated differently than at least one other layer, due, without limitation, to differences in drug concentration within different layers, absence or presence of the therapeutic agent and/or the auxiliary agent within different layers; differences in the means by which the drug(s) is associated with the polymer matrix in different layers, or the chemistry and density of the biodegradable materials.

Thus, release of an agent from the present implants can be related to the amount of a drug present in the implant and the properties of the polymers of the implant, such as polymer molecular weight and ratio of glycolide and lactide incorporated into the polymer. In one embodiment of the present implants, the drug or drugs, such as the antioxidant and/or auxiliary agent, is released at a first rate for a first time period that is substantially independent of the polymer properties, and the drug or drugs is released at a second rate for a second time period after the first time period that is dependent on the polymer properties of the implant. For example, an implant comprises a steroid and a polymeric component that releases the antioxidant from the implant for a time period of about thirty days primarily due to steroid dissolution, and releases the steroid from the implant after thirty days primarily due to polymer properties.

One example of the biodegradable intraocular implant comprises antioxidant (and/or auxiliary agent) associated with a biodegradable polymer matrix, which comprises a mixture of different biodegradable polymers. At least one of the biodegradable polymers is a polylactide having a molecular weight less than 40 kD. Such a mixture is effective in sustaining release of a therapeutically effective amount at least one agent(s) for a time period greater than about two months from the time the implant is placed in an eye. In certain embodiments, the polylactide has a molecular weight less than 20 kD. In other embodiments, the polylactide has a molecular weight of about 10 kD. The polylactide may be a poly(D,L-lactide), and the polylactide may include polymers having terminal free acid groups. In one particular embodiment, the matrix of the implant comprises a mixture of poly(lactide-co-glycolide) and polylactide. Each of the poly(lactide-co-glycolide) and polylactide may have terminal free acid groups.

Another example of a biodegradable intraocular implant comprises antioxidant (and/or auxiliary agent) associated with a biodegradable polymer matrix, which comprises a mixture of different biodegradable polymers, each biodegradable polymer having an inherent viscosity from about 0.15 dL/g to about 0.25 dL/g. For example, one of the biodegradable polymers may have an inherent viscosity of about 0.15 dL/g. Or, the mixture may comprise two different biodegradable polymers, and each of the biodegradable polymers has an inherent viscosity of about 0.25 dL/g.

Other implants may include a biodegradable polymer matrix of biodegradable polymers, at least one of the polymers having an inherent viscosity of about 0.25 dL/g to about dL/g. Additional implants may comprise a mixture of biodegradable polymers wherein each polymer has an inherent viscosity from about 0.50 dL/g to about 0.70 dL/g.

The release of the antioxidant (and/or auxiliary agent) from the intraocular implant comprising a biodegradable polymer matrix may include an initial burst of agent release followed by a gradual increase in the amount of the agent released, or the release may include an initial delay in release of the steroid followed by an increase in release. When the implant is substantially completely degraded, the percent of the agent that has been released is about one hundred. Compared to existing implants, in one embodiment the implants disclosed herein do not completely release, or release about 100% of at least one agent (antioxidant and/or auxiliary agent), until after about two months of being placed in an eye. Thus, the implants exhibit a cumulative release profile that may have a shallower slope, or a lower rate of release, for longer periods of time than existing implants.

Implants can provide therapeutically effective amounts of the antioxidant for prolonged

periods of time, or for a series of such time periods, without requiring such large doses. Thus, present implants may contain 1, 2, 3, 4, or 5 micrograms of antioxidant, the antioxidant is gradually released over time without causing substantial ocular toxicity or other adverse side effects. The antioxidant may, in one embodiment, be alternated within different shells of the implant such that it is delivered only over particular time periods, with time periods of substantially less (or no) antioxidant being delivered intervening. In this way continued exposure of the eye to the antioxidant, and the side effects that may accompany such constant delivery, may be avoided or reduced. In another embodiment, an intravitreal implant comprises antioxidant and a biodegradable polymer associated with the antioxidant in the form of an intravitreal implant that releases the antioxidant in amounts associated with a reduced toxicity relative to the toxicity associated with administering antioxidant in a liquid composition.

It may be in certain cases desirable to provide a relatively constant rate of release of the antioxidant from the implant over the life of the implant. For example, it may be desirable for the steroid to be released in amounts from about 0.01 μg to about 2 μg per day for the live of the implant. However, the release rate may change to either increase or decrease depending on the formulation of the biodegradable polymer matrix. In addition, the release profile of the steroid may include one or more linear portions and/or one or more non-linear portions. Preferably, the release rate is greater than zero once the implant has begun to degrade or erode.

It may be desirable to include delivery of an auxiliary agent in conjunction with the intravitreal or subconjunctival delivery of antioxidant in order to reduce or eliminate at least one side effect compared to the delivery of the steroid in an otherwise identical manner without the auxiliary agent. The auxiliary agent and antioxidant may be included in the same implant or co-administered in different implants during the same treatment period.

The implants may be monolithic, i.e., having the active agent or agents homogenously distributed through the polymeric matrix, or encapsulated, where the active agent is encapsulated by the polymeric matrix. Due to ease of manufacture, monolithic implants are usually preferred core/shell reservoir-type encapsulated forms. However, the greater control afforded by the encapsulated or reservoir-type implant may be of benefit in some circumstances, where the therapeutic level of the drug falls within a narrow window. In addition, the therapeutic component, including the antioxidant, may be distributed in a non-homogenous pattern in the matrix. For example, the implant may include a portion that has a greater concentration of the antioxidant and/or auxiliary agent relative to a second portion of the implant.

In another embodiment of the present invention, an intraocular implant comprises a therapeutic component, including an antioxidant, and an agent release sustaining component including one or more coating covering a core region of the implant. The therapeutic component and/or auxiliary agent is provided in the core region. The polymeric outer layer may be relatively impermeable to the therapeutic component and ocular fluids. Or, the polymeric outer layer may be initially impermeable to the therapeutic component and ocular fluids, but then may become permeable to the therapeutic component or ocular fluids as the outer layer degrades. Thus, the polymeric outer layer may comprise a polymer such as polytetrafluoroethylene, polyfluorinated ethylenepropylene, polylactide, polyglycolide, silicone, or mixtures thereof.

The foregoing implant may be understood to include a reservoir of one or more therapeutic agents, such as a antioxidant and/or auxiliary agent.

In some implants, the agent release sustaining component comprises a polymeric outer layer covering the therapeutic component and or the auxiliary agent, the outer layer comprises a plurality of openings or holes through which the therapeutic component may pass from the drug delivery system to an external environment of the implant, such as an ocular region of an eye. The holes enable a liquid to enter into the interior of the implant and dissolve the agent contained therein. The release of the therapeutic agent and/or auxiliary agent from the implant may be influenced by the drug solubility in the liquid, the size of the hole(s), and the number of holes. In certain implants, the hole size and number of holes are effective in providing substantially all of the desired release characteristics of the implant. Thus, additional excipients may not be necessary to achieve the desired results. However, in other implants, excipients may be provided to further augment the release characteristics of the implant.

Various biocompatible substantially impermeable polymeric compositions may be employed in preparing the outer layer of the implant. Some relevant factors to be considered in choosing a polymeric composition include: compatibility of the polymer with the biological environment of the implant, compatibility of the drug with the polymer, ease of manufacture, a half-life in the physiological environment of at least several days, no significant enhancement of the viscosity of the vitreous, lack of cytotoxicity, and the desired rate of release of the agent. Depending on the relative importance of these characteristics, the compositions can be varied. Several such polymers and their methods of preparation are well-known in the art. See, for example, U.S. Pat. Nos. 4,304,765; 4,668,506 4,959,217; 4,144,317, and 5,824,074, Encyclopedia of Polymer Science and Technology, Vol. 3, published by Interscience Publishers, Inc., New York, latest edition, and Handbook of Common Polymers by Scott, J. R. and Roff, W. J., published by CRC Press, Cleveland, Ohio, latest edition.

The polymers of interest may be nonbiodegradable, nonbioerodible, or nonbioabsorbable. The polymers of interest may be homopolymers, copolymers, straight, branched-chain, or cross-linked derivatives. Some exemplary polymers include: polycarbamates or polyureas, cross-linked poly(vinyl acetate) and the like, ethylene-vinyl ester copolymers having an ester content of 4 to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, or mixtures thereof.

Additional examples include polymers such as: poly(methylmethacrylate), polybutylnethacrylate, plasticized poly(vinyl chloride), plasticized polyamides) plasticized nylon, plasticized soft nylon, plasticized poly(ethylene terephthalate), natural rubber, silicone, polyisoprene, polyisobutylene, polybutadiene, polyethylene, polytetrafluoroethylene, poly(vinylidene chloride), polyacrylonitrile, cross-linked polyvinylpyrrolidone, chlorinated polyethylene, poly(trifluorochloroethylene), poly(ethylene chlorotrifluoroethylene), polytetrafluoroethylene, poly(ethylene tetrafluoroethylene), poly(4,4-isopropylidene diphenylene carbonate), polyurethane, polyperfluoroalkoxy) poly(vinylidene fluoride), vinylidene chloride-acrylonitrile copolymer, vinyl chloride-diethyl fumarate copolymer, silicone, silicone rubbers (of medical grade such as SILASTIC® Medical Grade ETR Elastomer Q7-4750 or Dow Corning® MDX 4-4210 Medical Grade Elastomer); and cross-linked copolymers of polydimethylsilanesilicone polymers.

Some further examples of polymers include: polydimethylsiloxanes, ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), polyvinyls such as poly(vinyl acetate), cross-linked polyvinylalcohol, cross-linked poly(vinyl butyrate), ethylene ethylacrylate copolymer, poly(ethyl hexylacrylate), poly(vinyl chloride), poly(vinyl acetals), plasticized ethylene vinyl acetate copolymer, polyvinylalcohol, poly(vinyl acetate), ethylene vinylchloride copolymer, polyvinyl esters, polyvinylbutyrate, polyvinylformal, polyacrylate, polymethacrylate, poly(oxides), polyesters, polyamides, and polycarbonates, or mixtures thereof.

In some aspects, the implants with an outer layer coating (with or without holes) may be biodegradable wherein the outer layer degrades after the drug has been released for the desired duration. The biodegradable polymeric compositions may comprise any of the above-identified biodegradable polymers or combinations thereof. In some implants, the polymer is polytetrafluoroethylene, (commercially known as TEFLON®), ethyl vinyl alcohol or ethylene vinyl acetate, all of which are nonbiodegradable, nonbioerodible or nonbioabsorbable.

The antioxidant-containing implants typically exhibited desirable release times with orifices configured to have a total area of less than 1% of the total surface area of the implant. A substantially cylindrically shaped implant has a first end, a second end, and a body portion between the first end and the second end. Typically, the implants disclosed herein are sealed at the first and second ends. One or more holes are formed in the body portion of the implant. The holes typically have a diameter of at least about 250 μm and less than about 500 μm. For example, holes may have a diameter of about 250 μm, 325 μm, 375 μm, or 500 μm. Smaller holes may be provided in other implants. Typically, two or three holes are provided in the implant outer layer. The holes may be spaced apart by a distance from about 1 mm to about 2 mm for implants having a length of about 7 mm to about 10 mm.

The implant may be capable of releasing the antioxidant at concentrations less than 2 μg/day. Some implants may be capable of releasing the antioxidant at a concentration of about 0.5 μg/day. Implants may be capable of providing therapeutically effective amounts of the antioxidant to an ocular region of an eye for more than one year, such as for more than five years, and even for about 10 years.

The implant may include a coating formed around a core containing a therapeutic agent. The core may include a therapeutic agent (or a therapeutic component and an auxiliary agent) associated with a biodegradable polymer matrix, or the core may be formed by filling a preformed coating, such as a tube.

Antioxidant and/or auxiliary agent can each be deposited in a preformed coating as a dry powder, particles, granules, or as a compressed solid. The agent or agents may also be present in a solution. In addition, the core can comprise a mixture of a biodegradable polymer matrix and the agent or agents, such as the matrix containing implants described above. The polymers used in the matrix with the therapeutic agent and/or auxiliary agent are bio-compatible with body tissues and body fluids and can be biodegradable or substantially insoluble in the body fluids. Any of the above-described biocompatible polymer compositions can be used to prepare the matrix. The amount of polymer in the core may be from about 0% to 80 wt % by weight. These polymers are commercially available and methods for preparing polymer matrices are well-known in the art. See, for example, U.S. Pat. No. 5,882,682.

The biocompatible, substantially impermeable outer layer can be obtained by coating the core with a polymeric composition described above. The coating can be applied using organic solvents, and the solvents may then be vacuum stripped from the coating to leave a dry coating. The polymer, at a concentration of from about 10 to about 80 weight percent is dissolved or suspended in an organic solvent at the appropriate temperature, for example for poly(lactic acid) or polylactide polymer, between 60 degrees to 90 degrees C. The resulting mixture can be cut, molded, injection molded, extruded, or poured or sprayed onto a pre-formed core into any shape or size for implantation. The spraying can be accomplished in a rotating pan coater or in a fluidized bed coater until the desired coating thickness is achieved.

Alternatively, the core may be dip coated or melt coated. This type of coating is especially useful with waxes and oils. In another embodiment, the core may be compression coated, wherein a suitable polymeric composition may be pressed onto a preformed core. In another aspect, an adhesive coat such as shellac or poly(vinyl acetate phthalate) (PVAP) is applied to the core prior to applying the impermeable coating in order to improve adhesion of the impermeable coating to the core. These techniques are well-known in the art. See, for example, Handbook of Common Polymers, by J. R. Scott and W. J. Roff, Section 64, (1971) published by CRC Press, Cleveland, Ohio.

When the outer layer is injection molded or extruded into the desired shape, the cavity formed by the outer layer can be then filled with the therapeutic agent and/or auxiliary agent composition. Then, the ends are sealed with an end cap. At least one orifice is drilled in the device. Optionally, an orifice is drilled, or preformed in the wall, or an orifice is sealed with a break-off tab that is broken open, or cut open, or the like, at the time of use.

Alternatively, the core-free device may be loaded with therapeutic agent by, for example, immersing the device in a solution comprising the therapeutic agent for a time sufficient for absorption of the therapeutic agent. The device may be equipped with a hollow fiber and the therapeutic agent and/or auxiliary agent may be directly loaded into the fiber and the device subsequently sealed. Where the activity of the therapeutic agent and/or auxiliary agent will not be compromised, the therapeutic agent-filled device may then be dried or partially dried for storage until use. This method may find particular application where the activity of the therapeutic agent of choice is sensitive to exposure to solvents, heat or other aspects of the conventional solvent-evaporation, molding, extrusion or other methods described above.

Holes may be made by drilling the appropriate size hole through a wall of the device using a mechanical or laser-based process. In some implants, a digital laser marking system is used to drill the holes. This system allows for an array of apertures to be drilled on both faces of a dosage form simultaneously and at rates suitable for production of dosage forms. The process utilizes a digital laser marking system (for example the DigiMark™ variable marking system, available from Directed Energy, Inc.) to produce an unlimited number of holes through the surface or coating of the dosage form, at rates practically suitable for production of dosage forms.

Steps involved in this laser drilling process are as follows: a digital laser marking system is focused at a later stage; the dosage form is moved onto the laser stage of the digital laser marking system is pulsed to energize those laser tubes needed to drill the desired apertures along a linear array on the dosage form, the dosage form is moved forward on the laser stage and the digital laser marking system is again pulsed as needed to produce an additional linear array of apertures; the dosage form is then removed from the later stage.

Orifices and equipment for forming orifices are disclosed in U.S. Pat. Nos. 3,845,770; 3,916,899; 4,063,064 and 4,008,864. Orifices formed by leaching are disclosed in U.S. Pat. Nos. 4,200,098 and 4,285,987. Laser drilling machines equipped with photo wavelength detecting systems for orienting a device are described in U.S. Pat. No. 4,063,064 and in U.S. Pat. No. 4,088,864.

Intraocular implants may have a size of between about 5 μm and about 10 mm, or between about 10 μm and about 1 mm, or about 0.4 mm to about 7 mm, or about 0.3 mm to about 7 mm or 8 mm, for administration with a needle, greater than 1 mm, or greater than 2 mm, such as 3 mm or up to 10 mm, for administration by surgical implantation. For needle-injected implants, the implants may have any appropriate length so long as the diameter of the implant permits the implant to move through a needle. For example, implants having a length of about 6 mm to about 7 mm have been injected into an eye. The implants administered by way of a needle should have a diameter that is less than the inner diameter of the needle. In certain implants, the diameter is less than about 500 μm. The vitreous chamber in humans is able to accommodate relatively large implants of varying geometries, having lengths of, for example, 1 to 10 mm. The implant may be a cylindrical pellet (e.g., rod) with dimensions of about 0.2 mm to 0.75 mm diameter. Or the implant may be a cylindrical pellet with a length of about 4 mm to about 6 mm or about 7 mm to about 10 mm, and a diameter of about 0.4 mm to about 0.75 mm to about 1.5 mm.

Implants may also be at least somewhat flexible so as to facilitate both insertion of the implant in the eye, such as in the vitreous, and accommodation of the implant. The total weight of the implant is usually about 100, 150, 150, 250 300, 333, 400, 500, 600, 700, 750, 800, 900, 1,000, 2,000, 2,500, 3,000, 4,000, to 5000 μg, more preferably about 500, 600, 700, 750, 800, 900, 1,000 μg. For example, an implant may be about 500 Ilg, or about 1000 μg. For non-human individuals, the dimensions and total weight of the implant(s) may be larger or smaller, depending on the type of individual. For example, humans have a vitreous volume of approximately 3.8 mL, compared with approximately 30 mL for horses, and approximately 60-100 mL for elephants. An implant sized for use in a human may be scaled up or down accordingly for other animals, for example, about 8 times larger for an implant for a horse, or about, for example, 26 times larger for an implant for an elephant.

Implants can be prepared where the center may be of one material and the surface may have one or more layers of the same or a different composition, where the layers may be cross-linked, or of a different molecular weight, different density or porosity, or the like. For example, where it is desirable to quickly release an initial bolus of drug, the center may be a polylactide coated with a poly(lactide-co-glycolide) copolymer, so as to enhance the rate of initial degradation. Alternatively, the center may be poly(vinyl alcohol) coated with polylactide, so that upon degradation of the polylactide exterior the center would dissolve and be rapidly washed out of the eye.

Implants, particularly the implants with the antioxidant and/or auxiliary agent associated with a biodegradable polymer matrix, may be of any geometry including fibers, sheets, films, microspheres and microparticles, spheres, circular discs, plaques and the like. The upper limit for the implant size will be determined by factors such as toleration for the implant, size limitations on insertion, ease of handling, etc. Where sheets or films are employed, the sheets or films will be in the range of at least about 0.5 mm×0.5 mm, usually about 3-10 mm×5-10 mm with a thickness of about 0.1-1.0 mm for ease of handling. Where fibers are employed, the fiber diameter will generally be in the range of about 0.05 to 3 mm and the fiber length will generally be in the range of about 0.5-10 mm. Spheres may be in the range of about 0.5 μm to 4 mm in diameter, with comparable volumes for other shaped particles.

In certain embodiments of the present invention the use if microparticle implants may be particularly advantageous. A method of making such microparticles involves combining, associating, or mixing, the therapeutic and/or auxiliary agent with a biodegradable polymer or polymers. The mixture may then be extruded or compressed to form a single composition. The single composition may then be processed to form microparticles suitable for placement intravitreally or subconjunctivally.

Alternatively, a method of making the present microparticles may also include using an oil-in-water emulsion process to form the microparticles. Such methods may be particularly useful in forming microparticles, nanoparticles and the like. Thus, an embodiment of the present invention relates to the inserts comprising microparticles made using an oil-in-water emulsion process.

A population of microparticles or nanoparticles, may be placed in an ocular region such as, without limitation, intravitreal, intrastromal, intracameral, subtenon, retinal, subretinal, retrobulbar, peribulbar, suprachoroidal, subchoroidal, conjunctival, subconjunctival, episcleral, posterior juxtascleral, anterior juxtascleral, circumcorneal, topical, and/or tear duct, to treat a variety of ocular conditions. For example, the microparticles may be administered intravitreally in a manner effective to delivering a therapeutic component and/or auxiliary agent to tissues of the posterior segment, thereby reducing damage to the tissues of the posterior segment while reducing at least one side effect as compared to the administration of the steroid alone in an otherwise identical manner. Alternatively, subconjunctival administration of the microparticles of the present invention are very effective at delivering the therapeutic component to the retina and other tissues of the posterior segment for the treatment of neurodegenerative conditions such as age-related macular degeneration (AMD), such as “wet” or “dry” AMD, macular edema, etc.

Use of microparticles (or microspheres) and the like provides an excellent means of punctuated delivery of the antioxidant in the implants of the present invention. For example, in one embodiment different lots (comprising the same or different sizes of microparticles) are made, each having a different property, such as different rates of erosion; different drug content (for example some may contain an antioxidant and an auxiliary agent, while others may just contain the auxiliary agent; some may be made of one bioabsorbable polymer having a fast dissolution rate, while others may be made of a different biopolymer having a slower dissolution rate. By engineering the microparticles so that during the treatment period the dosage of antioxidant is “pulsed”, for example, from an initial substantially optimal therapeutically effective dosage to a subsequent period lacking a substantially optimal therapeutically effective dosage of the antioxidant and optionally to another treatment time period in which a substantially optimal therapeutically effective dosage of the antioxidant is again administered, at least one of the deleterious side effects of long term antioxidant use can be lessened. Some microparticles may, for example, be loaded with the auxiliary agent, either alone or in combination with the antioxidant, to provide a substantially constant (or at least slowly decaying) dosage of the auxiliary agent to ocular tissues during the treatment period, while the dosage of antioxidant may vary.

Thus, the combination of different microparticles in a discretely administered intravitreal or subconjunctival injection or insertion provides a powerful way to separately tailor the administration of antioxidant and auxiliary agent. Methods of making microparticles are provided in U.S. application Ser. No. 11/303,462, and U.S. application Ser. No. 10/837,260.

Size and form of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

Proportions of antioxidant and/or auxiliary agent, polymer, and any other modifiers may be empirically determined by formulating several implants with varying proportions. A USP approved method for dissolution or release test can be used to measure the rate of release (USP 23; NF 18 (1995) pp. 1790-1798). For example, using the infinite sink method, a weighed sample of the implant is added to a measured volume of a solution containing 0.9% NaCl in water, where the solution volume will be such that the drug concentration is after release is less than 5% of saturation. The mixture is maintained at 37° C. and stirred slowly to maintain the implants in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

The antioxidant included in the intraocular implants disclosed herein, the intraocular implants may also include one or more additional ophthalmically acceptable therapeutic agents.

The implant is injected through the pars plana into the vitreous of each of the patient's eyes using an applicator with a 22-gauge needle. The patient is monitored following the administration of the implant.

### EXAMPLE 1. diNACA INHIBITIONOF CATARACT IN RATE LENS METHOD

Lenses were extracted from 3-week-old male or female Wistar rats using an existing Animal Ethics Approval for tissue collection. Rats were euthanized using CO₂, eyes were enucleated and lenses dissected from the globe and incubated in either Dulbecco's modified eagle's medium (low glucose; DMEM; Savion et al., 2019) or Media 199, no phenol red with Glutamine, with 1% penicillin, streptomycin, neomycin mixture for 6 hours, under normoxic conditions with 5% CO₂. At the end of the 6-hour incubation, lenses were imaged under darkfield and brightfield microscopy and only clear, transparent lenses were used for further experimentation.

Control group: Lenses were incubated for a further 18 hours at which time, darkfield and brightfield images were taken. Lenses were placed in fresh media and then incubated for another 24 hours in DMEM or M199 with 1% PSN. Images were then taken to assess lens transparency.

10 mM diNACA pre-treatment group: After the initial 6-hour incubation, clear lenses were cultured in 10 mM diNACA (NPI-002) for 18 hours. Images were taken, and then lenses were placed in fresh media containing 1 μg/mL of glucose oxidase (GO). Lenses were then incubated for a further 24 hours, after which images were taken to assess lens transparency

10 mM NACA pre-treatment group: After the initial 6-hour incubation, clear lenses were cultured in 10 mM NACA (NPI-001) for 18 hours. Images were taken, and then lenses placed in fresh media containing 1 μg/mL of GO. Lenses were then incubated for a further 24 hours, after which images were taken to assess lens transparency

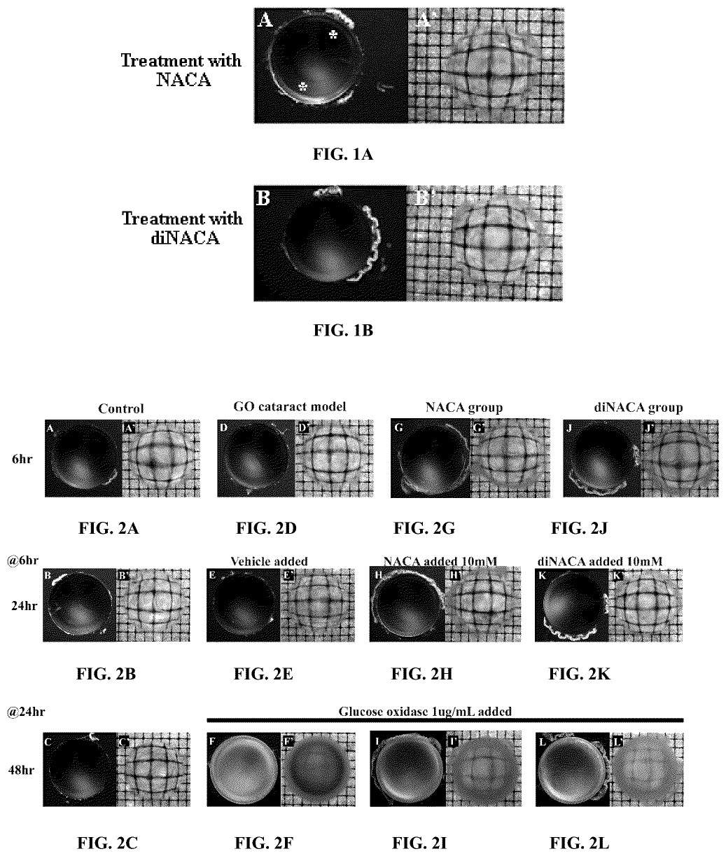

Results. The addition of 1 μg/mL of glucose oxidase (GO) after 24 hours produced a cataract by 48 hours (some of the gridlines were barely visible from brightfield images (FIG. 1). NPI-002 (diNACA) alone, with no oxidative agent, maintained lens transparency slightly better than NPI-001 (NACA) alone. At 6 hours, clear transparent lenses were assigned to either no treatment, or 10 mM DiNACA for 24 hours. NACA-treated lenses developed a subtle thin rim opacity on the edge (FIG. 1A-opacities are marked by *). However, the underlying grid was clearly visible whereas surprisingly diNACA-treated lenses remained absolutely clear and transparent (FIG. 1B).

### EXAMPLE 2. NACA or diNACA PRE-TREATMENT OF LENSERS IN A GO-CATARACT MODEL REDUCED LENS OPACITIES

Method: Isolated rat lenses were pre-treated with NACA or diNACA followed by GO.

Results: Pre-treatment with 10mM NACA (NPI-001), followed by GO-cataract induction, resulted in reduced opacities compared to cataract induction alone (n=8; FIGS. 2A to 2L, compare panel F to I, and F′ to I′). The opacity appears reduced in the NACA group compared to the GO group (FIGS. 2A to 2L, compare panel 2F to 2I), and the underlying grid is more clearly visible in the NACA group compared to the GO group (FIGS. 2A to 2L, compare panel 2F′ to 2I′). These observational differences suggest that pre-treatment with 10 mM NACA may help to reduce lens opacities caused by GO. Pre-treatment with 10 mM diNACA, followed by GO-cataract induction, also reduced lens opacities (n=7; FIGS. 2A to 2L, compare panel 2F and 2L) and improved grid visibility (FIGS. 2A to 2L, compare panel F′ and L′) compared to GO-cataract induction alone.

Conclusions: Rat lenses tolerated exposure to diNACA better than NACA as a slight ring opacity developed following exposure to NACA that did not develop with diNACA.

### EXAMPLE 3: diNACA INHIBITION OF CATARACTS IN ISOLATED PORCINE LENS

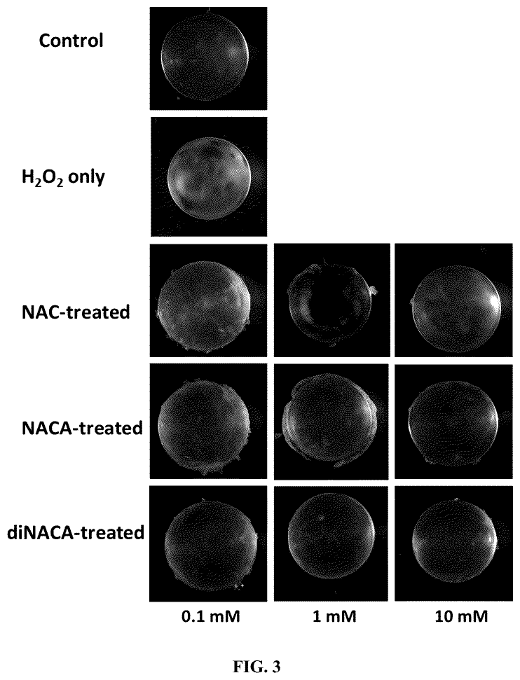

Method: Lenses were harvested from fresh eyes from pigs. These lenses were first incubated in 4-5 mL TC-199 medium and 1% pig serum in a 12-well plate for two hours. At the end of the incubation, lenses were imaged under darkfield microscopy and only clear, transparent lenses were used for further experimentation. Lens were pre-incubated with NAC, NACA, diNACA for 24 hours. After the pretreatment, the lenses were transferred to media containing hydrogen peroxide (H₂O₂) plus GO for maintaining a constant H₂O₂ level during incubation similarly to a published method (Wang et al., 1997). At the end of the 6-hour incubation, lenses were imaged under darkfield microscopy (FIG. 3).

Results and Conclusions. NAC, NACA and diNACA pretreatment dose-dependently prevented H₂O₂-induced cataract, suggesting that these compounds may have anti-cataract potential. As compared with NAC, NACA, surprisingly diNACA had better protective effects on the lens.

### EXAMPLE 4: SUMMARY OF diNACA ANTICATARACT EFFECTS IN EX VIVO LENS MODELS

Anticataract effects of NPI-002 isolated rat lens and isolated porcine lens are

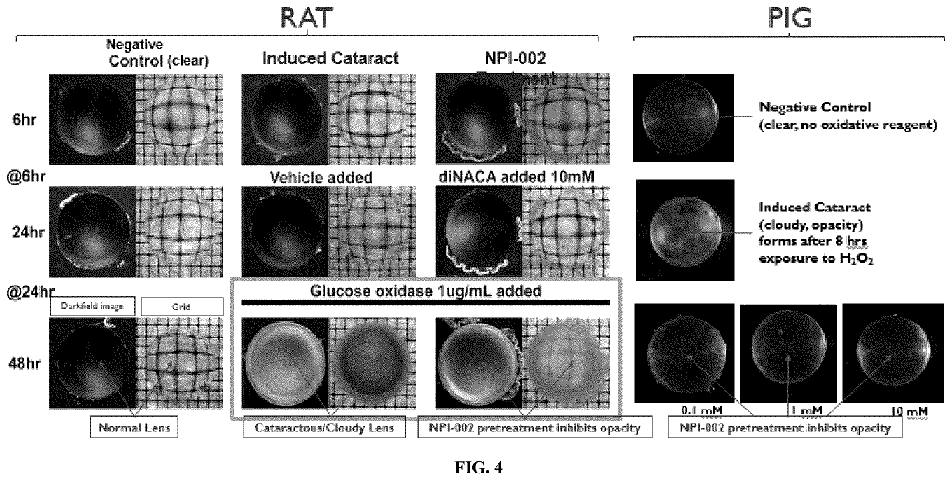

comparable (FIG. 4). NPI-002 prevented oxidative-induced cataract caused by two different oxidative reagents (GO in rat; H₂O₂ in pig), in two species, in studies by two independent research groups.

### EXAMPLE 5. NPI-002 INTRAVITREAL (IVT) IMPLANT SINGEL DOSE 3-WEEK SAFETY AND PHARMACOKINETIC STUDY IN RABBIT

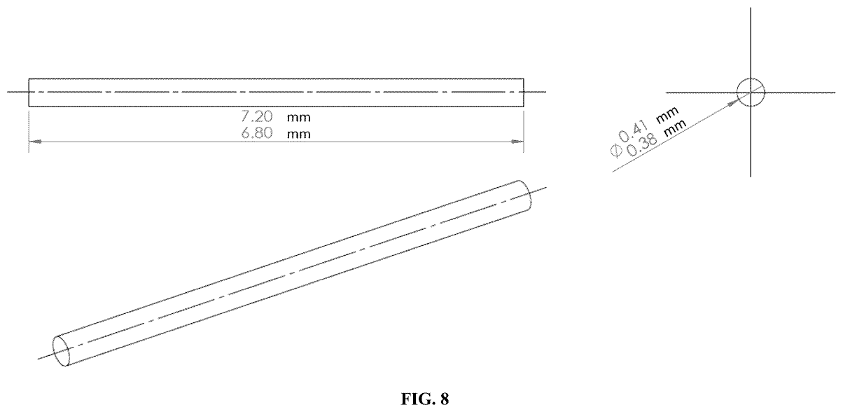

A study was conducted to evaluate intravitreal injection of di-NACA as suspension or implant and NACA solution in rabbit. The objectives were to assess safety and whether the implant could deliver diNACA, NACA, and NAC to ocular tissues. A sterile intravitreal implant containing diNACA was prepared (Formulation 1896-08, a fast-releasing implant; FIG. 7). Female New Zealand White rabbits were administered two NPI-002 IVT implants per eye (total dose of NPI-002, 0.8 mg/eye). One group of animals received IVT placebo implants without any test article. FIG. 8 shows dimensions of implant.

Prior to placement on study, an ophthalmic examination was conducted on each rabbit including slit-lamp biomicroscopy and indirect ophthalmoscopy. Ocular findings were scored according to a modified McDonald-Shadduck Scoring.

Clinical ophthalmic examinations were performed at baseline and on Days 8 and 23 post-dose. General health observations were performed daily. Body weights were recorded at baseline and prior to termination.

Subjects were assigned as shown in Table 1. Sterile NPI-002 or placebo IVT implants were provided pre-loaded in 22 G beveled needle with wire plunger (1 implant/needle) and injected into VH. NPI-002 suspension and NPI-001 solution were prepared in sterile phosphate buffer and injected into VH.

Animals were euthanized on Day 8, 15, or 23, and ocular tissue was collected and

submitted for bioanalysis for diNACA, NACA and NAC. Delivery of IVT di-NACA was associated with minimal ocular findings on the day of dosing. Observations of conjunctival bulging, discharge, and minimal hemorrhaging were determined to be related to the injection procedure and resolved in most eyes on the day after dosing. Mild to moderate conjunctival congestion, swelling and discharge observed on Day 8 by clinical ophthalmic examinations were resolved by Day 23, suggesting that these findings were associated with the use of 22-gauge needle for IVT injection procedure.

Delivery of di-NACA as suspension or implant, placebo implant, and NACA as solution via IVT injection was associated with minimal ocular findings on the day of dosing. Observations of conjunctival bulging, discharge, and minimal hemorrhaging were determined to be related to the injection procedure and resolved in most eyes on the day after dosing.

TABLE 1
Group Assignments

| | | | Dose | | | | | | | |
|---|---|---|---|---|---|---|---|---|---|---|
| | | | Route | | | Dose | | | | |
| | | | and | | | Volume | | Clinical | | Matrices |
| | | Treatment | Regimen | Dose | | or | | Ophthalmic | | Collected |
| Group | N | (OU) | (OU) | (mg/eye) | (mmol/eye) | Amount | Concentration | Examinations | Termination | (OU) |
| 1 | 15 | NPI- | IVT on | ~0.8 | 0.0025 | 2 | ~0.4 mg | Baseline | Days | Plasma, |
| | | 002 | Day 0 | (2 × ~0.4) | (2 × 0.00125) | implants | per implant | & | 8, 15, | AH, |
| | | Implants | | | | per eye | | Termination | and | Lens, |
| | | | | | | | | | 23 | VH, |
| | | | | | | | | | | Retina, |
| 2 | 3 | NPI- | | 0.8 | 0.0025 | 50 | 16 mg/mL | | | & |
| | | 002 | | | | uL/eye | | | | Choroid/ |
| | | Suspension | | | | | | | | RPE |
| 3 | 5 | Placebo | | NA | NA | 2 | NA | | | |
| | | Implants | | | | implants | | | | |
| | | | | | | per eye | | | | |
| 4 | 3 | NPI- | | 0.4 | 0.0025 | 50 | 8 mg/mL | | | |
| | | 001 | | | | uL/eye | | | | |
| | | Solution | | | | | | | | |

OU: both eyes;
IVT: intravitreal injection;
NA: not applicable;
AH: aqueous humor;
VH: vitreous humor;
RPE: retinal pigment epithelium
#Group 1: N = 5 per time point;
Groups 2 and 4: N = 1 per time point;
Group 3: N = 1 on Day 7(±1) and N = 2 on Days 14(±1) and 21(±2).

Mild to moderate conjunctival congestion, swelling and discharge observed on Day 8 by clinical ophthalmic examinations were resolved by Day 23, suggesting that these findings were associated with the use of large 22 G needle for IVT injection procedure. In addition, in three eyes that received di-NACA implants, choroidal/retinal inflammation was also observed due to the implant hitting the retina and were also procedure related. Vitreal cells and flare detected on Day 8 and Day 23 were likely test article-related particles as these were mostly cleared, or present only in small numbers, in the vitreous humor on Day 23 compared to Day 8. A few of these reflective particles were attached to the posterior lens capsule.

No adverse effects of test article administration on general, non-ocular health, or on body weights were observed.

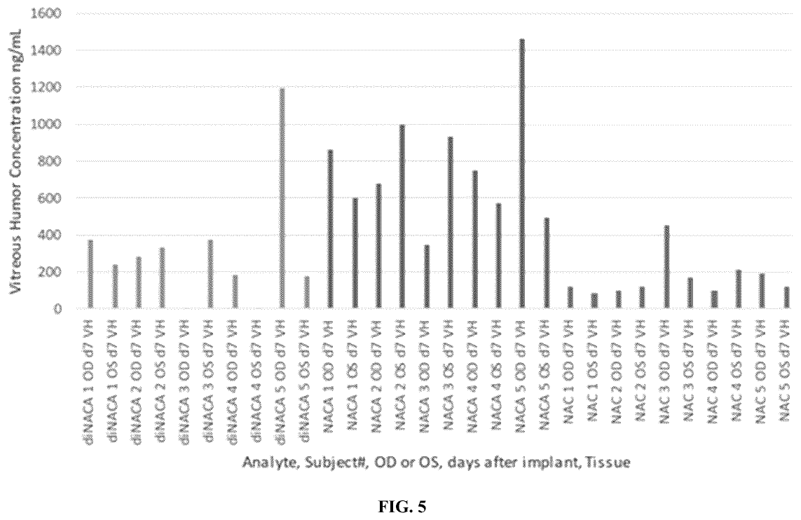

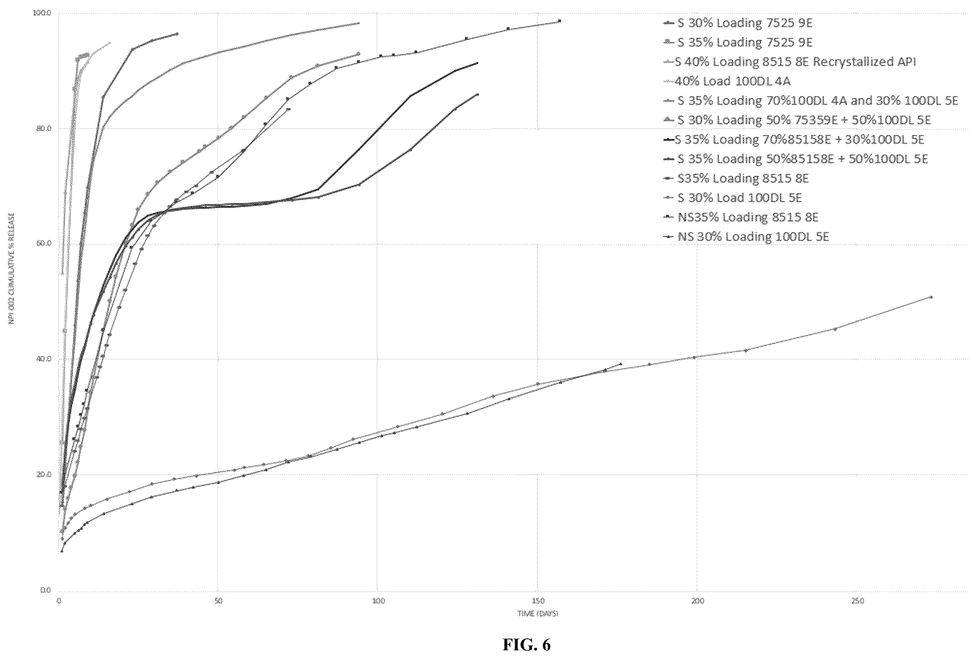

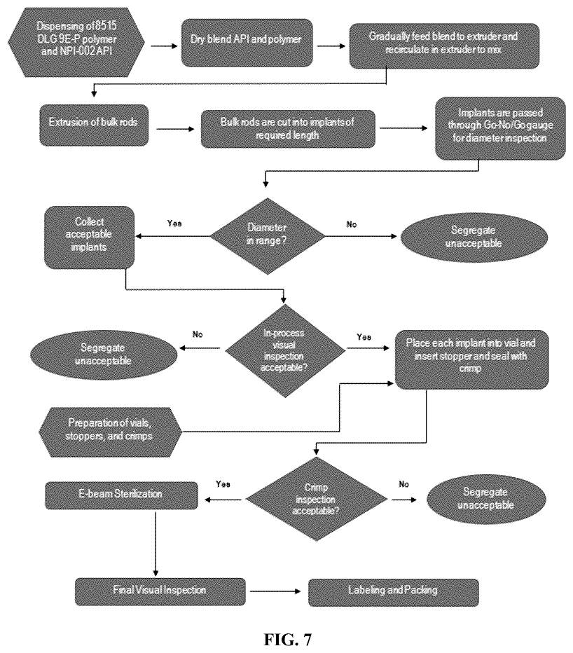

Intravitreal delivery of NPI-002 implant resulted in significant levels of diNACA, NACA, and NAC in the vitreous humor after 1 week (FIG. 5).

In summary, delivery of NACA solution and di-NACA as suspension or implant or placebo implant were generally well tolerated when administered via Intravitreal (IVT) injection.

Intravitreal delivery of NPI-002 implant resulted in significant levels of diNACA, NACA and NAC in the rabbit vitreous humor after 1 week, thereby demonstrating that diNACA is a prodrug to its major metabolites and pharmacologically active species, NACA and NAC.

### EXAMPLE 6. NPI-002 INTRAVITREAL (IVT) IMPLANT SINGLE-DOSE 3-MONTH TOXICOLOGY STUDY IN RABBIT

A study was conducted to evaluate safety of diNACA (NPI-002) IVT implant. The study comprised four groups of six rabbits (NZ White): (1) an untreated control group; (2) a drug-treated group that had one fast release NPI-002 implant injected in each eye; (3) a drug-treated group that had one fast release plus one slow release NPI-002 implant injected in each eye (FIG. 6); (4) a placebo group that had one placebo implant injected in the left eye and two placebo implants injected in the right eye. Clinical ophthalmic examinations were conducted at baseline, and, at 1 day, 1 week, 1 month, and 3 months after implant delivery. In addition, fundus imaging, spectral domain optical coherence tomography (SD-OCT) and electroretinography was performed at baseline and 3 months. Blood samples were collected. For groups 1-3, the left eyes were dissected, fixed, and processed for histology and immunohistochemistry to assess safety/toxicity. The right eyes were dissected and used for bioanalytical analyses. For group 4, the left and right eyes were dissected, fixed, and processed for histology and immunohistochemistry.

Overall, the implant injections proceeded with negligible complications. Overall health and weight of all 24 rabbits was satisfactory throughout the duration of the study. The most commonly encountered welfare incidents related to minor wounds encountered from momentary aggression between rabbits. All rabbits reached the designated endpoint of the study at 3 months. Blood samples were taken for future analysis. Of the 48 eyes, 30 eyes were taken for post-mortem histopathology, while 18 eyes were dissected for future pharmacokinetic analysis. No adverse effects of NPI-002 implants were observed on general health. Body weight gain was normal. There was no evidence from clinical ophthalmic examinations that NPI-002 implants caused detrimental effects to the anterior or posterior compartments of the eye, either acutely or chronically. There was no evidence from rebound tonometry that NPI-002 implants caused an alteration to intraocular pressure. There was no evidence from electroretinography that NPI-002 implants deleteriously affected the electrical function of the retina. There was no evidence from fundus imaging or SD-OCT scans that NPI-002 implants caused detrimental effects to the morphology of the retina. Overall, no differences were observed between untreated control, placebo-injected, and NPI-002-injected cohorts in terms of the various parameters assessed. No differences were observed between animals that received either one or two NPI-002 implants.

This study demonstrated the safety of one or two NPI-002 implants in the vitreous cavity of albino rabbits. No drug-related toxicity at the clinical, retinal imaging, electroretinographic or histological level was noted. These findings provide reassurance to proceed with a human Phase 1/2 clinical trial to investigate the safety and efficacy of one or two intravitreal NPI-002 implants per eye in attenuation of vitrectomy-induced cataract.

### EXAMPLE 7. NPI-002 REPEATED DOSE TOPICAL OCULAR ADMINISTRATION IN RABBIT

A study was conducted to evaluate effects of diNACA or NACA topical ocular dosing in rabbit. Ophthalmic formulations were developed containing either 1% diNACA or 1% NACA that were used for repeated topical ocular instillation to Dutch Belted rabbits. Three animals per group were dosed four times a day at approximately 8:00 AM, 11:00 AM, 2:00 PM, and 4:00 PM on Days 1-6, and once on Day 7 at approximately 8:00 AM, via bilateral topical administration with each test article. Ocular tolerability was assessed at baseline and prior to the first daily dose by means of scoring chemosis, discharge, and hyperemia (Draize scoring). Ocular tissue was harvested at necropsy (Day 7). A qualified analytical LC-MS method was developed and used to quantitate NACA levels in aqueous humor (AH). No serious adverse findings were observed following topical ocular dosing.

Mild discharge was observed at low frequencies in all groups, and mild hyperemia was observed in the vehicle group. No indications of chemosis were seen in any of the administration groups. Because the vehicle treatment showed similar or greater frequency of mild ocular irritation indicated by discharge or hyperemia, the ocular irritation seen in the test agent administration groups are possibly not attributable to the test agent themselves, rather to the vehicle. Levels of NACA were observed achieved in aqueous humor following dosing of either 1% diNACA or 1% NACA (Table 2). The presence of diNACA in the AH demonstrated that diNACA penetrates the eye following topical ocular dosing. The presence of NACA in the AH demonstrated that diNACA served as a prodrug to deliver NACA to the AH. It is unclear whether diNACA is cleaved to NACA on the ocular surface or after penetration into the AH.

TABLE 2
Levels of NACA Achieved in Aqueous humor Following
Topical Dosing in Rabbits of Either 1% diNACA or 1% NACA

| | | | | NACA in Aqueous |
|---|---|---|---|---|
| Subject Number | Arm | Treatment | Eye | Humor (ng/ml) |
| 1337 | 1 | 1% NACA | Left | 51.4 |
| | 1 | 1% NACA | Right | 23.7 |
| 1338 | 1 | 1% NACA | Left | 64.9 |
| | 1 | 1% NACA | Right | BLQ < (20.0) |
| 1339 | 1 | 1% NACA | Left | 33.5 |
| | 1 | 1% NACA | Right | BLQ < (20.0) |
| 1340 | 2 | 1% diNACA | Left | 56.3 |
| | 2 | 1% diNACA | Right | BLQ < (20.0) |
| 1341 | 2 | 1% diNACA | Left | 33.7 |
| | 2 | 1% diNACA | Right | 47.7 |
| 1342 | 2 | 1% diNACA | Left | 85.4 |
| | 2 | 1% diNACA | Right | 35.3 |

BLQ: below limit of quantitation

### EXAMPLE 8: MANUFACTURE OF IMPLANTS

NPI-002 IVT implant manufacture process flow is presented in FIG. 7.

A description of the IVT implant manufacturing process follows. Using a Fisher Pharma Mini Twin-Screw Extruder, the temperature of the feed throat chiller is set at 17° C. and the temperatures of Zone 1 and 2 are both set at 127° C. With the extruder speed set at 5 rpm, a NPI-002 and 8515 9E-PLG blend is added to the extruder feed funnel. Note: Blending of NPI-002 and 8515 9E-PLG is done previously using a Turbula blender (5-minute blending at a speed setting of 46). Once all of the NPI-002 and 8515 9E-PLG blend is added to the extruder, the screw rpm is increased to 15 and the NPI-002/8515 9E-PLG material is recirculated in the extruder for 10 to 12 minutes. Then with screw set at 5-10 rpms, the extrudate is allowed to pass through the extruder die. As the extrudate exits the die, it is carried by a conveyor through a Beta Lasermike to control the extrudate diameter to around 0.38 to 0.41 mm and collected as bulk rods. The bulk rods are then cut into IVT implant lengths.

A drug content method is used to determine the amount of NPI-002 and impurities in the implants. NPI-002 implants are incubated in acetonitrile to dissolve the PLG. Then diluent is added to extract the NPI-002. The extracted NPI-002 is analyzed against a reference standard of NPI-002 using a reversed phase, gradient HPLC method with UV detection at 214 nm. HPLC conditions are shown in Table 3.

TABLE 3
HPLC Conditions

| Parameter (nominal in parentheses) | Variation |
|---|---|
| Column temperature (30° C.) | 32° C. |
| | 28° C. |
| Flow rate (1.0 mL/min) | 1.2 mL/min |
| | 0.8 mL/min |
| Detector Wavelength (214 nm) | 217 nm |
| | 211 |

### EXAMPLE 11. CLINICAL STUDY OF NPI-002 INTRAVITREAL IMPLANT IN POST-VITRECTROMY SUBJECTS

Since vitrectomy surgery is a common procedure that causes rapid and reliable opacification of the lens nucleus, patients undergoing vitrectomy offer a promising ‘model system’ to test this hypothesis. Success in protecting post-vitrectomy patients from developing nuclear cataracts would lay the groundwork for interventions to prevent these cataracts in subjects with advanced vitreous degeneration (Beebe et al., 2010).

### Prophetic Example. NPI-002 Intravitreal Implant for the Delay of Cataract Progression

### Study Description.

Brief Summary: This study will examine the safety and efficacy NPI-002 intravitreal implants post vitrectomy.

| Condition or | | |
|---|---|---|
| disease | Intervention/treatment | Phase |
| Cataract | Drug: NPI-002 Intravitreal Implant | Phase 1/Phase 2 |

### Study Design:

Study Type: Interventional (Clinical Trial)
Estimated Enrollment: 30 participants
Allocation: Randomized
Intervention Model: Parallel Assignment
Masking: Double (Participant, Outcomes Assessor)
Primary Purpose: Prevention
Official Title: Safety and Efficacy of NPI-002 Intravitreal Implant for the Delay of Cataract Progression in Patients Undergoing Vitrectomy

### Arms and Interventions:

| Arm | Intervention/treatment |
|---|---|
| Experimental: Single NPI-002 | Drug: NPI-002 Intravitreal Implant |
| Intravitreal Implant | Implant inserted during vitrectomy. |
| Experimental: Double NPI-002 | Drug: NPI-002 Intravitreal Implant |
| Intravitreal Implant | Implant inserted during vitrectomy. |
| No Intervention: Control | No implant |

### Outcome Measures

### Primary Outcome Measures:

1. Lens Densitometry [Time Frame: 6 months]
   Change from Baseline
      Ages Eligible for Study: 18 Years and older
      Sexes Eligible for Study: All
      Accepts Healthy Volunteers: No

### Criteria

### Inclusion Criteria:

1. Indicated for vitrectomy
2. Natural Lens in study eye at time of vitrectomy
3. Some cataract present as assessed pre-operatively

### Exclusion Criteria:

1. Previous intraocular surgery in study eye
2. Clear zonular weakness or defects/coloboma
3. Not on stable dose of medications for other conditions
4. Anticipated need for oral corticosteroids during study participation
5. Evidence or history of uveitis or ocular ischemia (ischemic retinopathy)
6. Evidence or history of proliferative diabetic retinopathy
7. Previous panretinal photocoagulation procedure
8. Moderate or severe uncontrolled glaucoma
9. Current smoker
10. Use of supplemental oxygen
11. Current lung disease resulting in decreased oxygen saturation.
12. Sensitivity to thiol compounds.
13. Participation in another clinical trial.

### EXAMPLE 12. RABBIT STUDY OF diNACA INTRAVITREAL IMPLANT

This example shows the measurement of NAC, NACA, di-NACA and GSH in plasma samples taken at baseline as well as 3 months after implant delivery. Further, the measurement of NAC, NACA, di-NACA and GSH in aqueous humor, vitreous humor, lens, retina and RPE/choroid at 3 months after implant delivery was also determined.

TABLE 4
Treatment groups.

| | | fast | slow | |
|---|---|---|---|---|
| Group | treatment | release | release | placebo |
| 1 | Untreated (control) | — | — | — |
| 2 | NPI-002 (one implant each eye) | 1 | — | — |
| 3 | NPI-002 (two implants each eye) | 1 | 1 | — |
| 4 | placebo (one implant left eye; | — | — | 1 |
| | two implants right eye) | — | — | 2 |

n = 6 rabbits per group, both eyes are treated

Blood collection. Blood was not taken from animals in Group 1 (control). Blood was collected from each animal in groups 2, 3, and 4 at the time of implant delivery and again at the endpoint of the study (3 months post-implant delivery). Blood was placed into pre-chilled tubes containing K2EDTA as the anticoagulant, inverted several times to mix, and kept on wet ice until centrifugation. The blood samples were centrifuged at a temperature of 4° C., at approximately 3,000×g, for 5 minutes. The resultant plasma (at least 100 μL) was separated within approximately 30 minutes after centrifugation, collected into pre-labeled polypropylene tubes, snap-frozen on dry ice, and stored in a freezer set to maintain -60° to -80° C. Prior to snap freezing, the plasma was acidified with formic acid to stabilize the NPI-002 (0.5% by plasma volume).

Tissue Collection. Tissues harvested for pharmacokinetic evaluations.

The right eye was harvested from each rabbit in group 1 (control), group 2 (NPI-002, one implant), and group 3 (NPI-002, two implants). Both eyes from group 4 (placebo) were used for histological evaluations and were not available for pharmacokinetic analysis.

The following tissues were dissected: Aqueous humor (AH); Vitreous humor (VH); Lens; Retina; and Choroid/retinal pigment epithelium (RPE).

Samples from each eye were separated and were not pooled. Samples were collected into individual, pre-labeled, pre-weighed polypropylene tubes. The weight of each sample was recorded. The AH and VH (but not the other ocular tissues) were acidified with formic acid to stabilize the NPI-002 (0.2% by volume for humors). Samples were flash frozen in liquid nitrogen and placed on dry ice until storage in a freezer set to maintain −60 to −80° C. All samples were shipped frozen on dry ice to the Sponsor's designated laboratory, Agilex, for analysis. A total of 18 AH, 18 VH, 18 lens, 18 retina, and 18 choroid/RPE samples were collected and submitted for analysis.

Analyte concentrations in plasma. The NAC plasma level in the placebo group was 43.6 ng/mL at baseline and 38.2 ng/mL at 3 months after implant delivery. NAC plasma levels in both implant groups were approximately 65 ng/mL at baseline. No elevation in plasma NAC was detected in either group at 3 months after implant delivery. In fact, the NAC level was somewhat lower in both groups. NACA was essentially undetectable in all groups both at baseline and 3 months after implant delivery. The level of di-NACA was extremely low in all groups both at baseline and 3 months after implant delivery.

The GSH plasma level in the placebo group was 9,782 ng/ml at baseline and 18,270 ng/ml at 3 months after implant delivery. The GSH plasma levels in the single and double implant groups were 10,427 ng/mL and 6,513 ng/ml, respectively, at baseline, and 9,747 ng/mL and 10,377 ng/mL, respectively, at 3 months after implant delivery.

These data demonstrate that intravitreal implants containing di-NACA did not elevate plasma levels of NAC, NACA, di-NACA or GSH when analyzed 3 months after surgery. While statistically significant differences between baseline versus 3-month data were found in some parameters, such results are unlikely to represent genuine cause-effect relationships.

TABLE 5
Metabolite concentrations in plasma.

| | | Placebo | one implant | Two implants |
|---|---|---|---|---|
| metabolite | time | (group 4) | (group 2) | (group 3) |
| NAC | baseline | 43.6 | 65.6 | 65.8 |
| | 3 months | 38.2 | 48.8^{†} | 49.4 |
| NACA | baseline | 0 | 2.13 | 0 |
| | 3 months | 0 | 0 | 0 |
| di-NACA | baseline | 0.11 | 0.40 | 0.20 |
| | 3 months | 0.08 | 0.01^{†} | 0.03 |
| GSH | baseline | 9782 | 10427 | 6513 |
| | 3 months | 18270 | 9747 | 10377^{†} |

All concentrations represent ng/mL.
^{†}P < 0.05 by Student's paired t-test (baseline vs 3 months)
Metabolite concentrations in liquid and solid matrices.

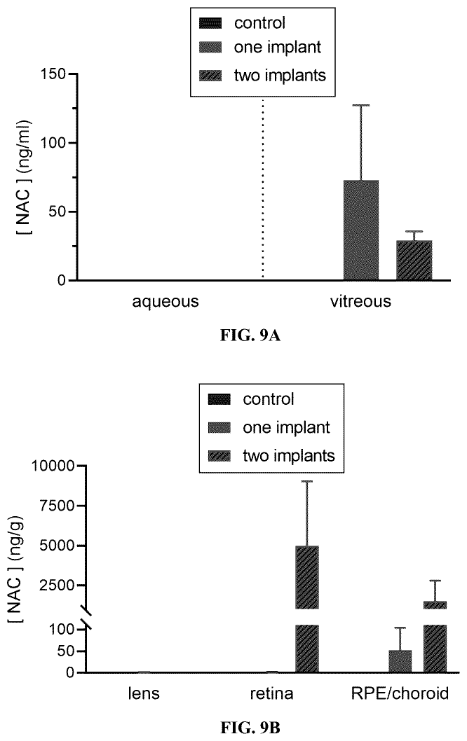

NAC: As shown in FIG. 9A, NAC was undetectable in aqueous humor in all three groups. In vitreous humor, NAC was undetectable in the control group, but was present at low levels (typically below the assay lower limit of quantification) in samples from both treated groups.

As shown in FIG. 9B, NAC was undetectable in the lens samples in all three groups. In the retina, NAC was essentially undetectable in the untreated and single (fast release) implant groups, but was highly elevated in the dual implant group, albeit that there was great variability between samples. In the RPE/choroid, NAC was undetectable in the untreated group, and undetectable in all but one sample of the single (fast release) implant group, but was highly elevated in the dual implant group, albeit not to the magnitude measured in the corresponding retinal samples. Again, there was great variability between animals.

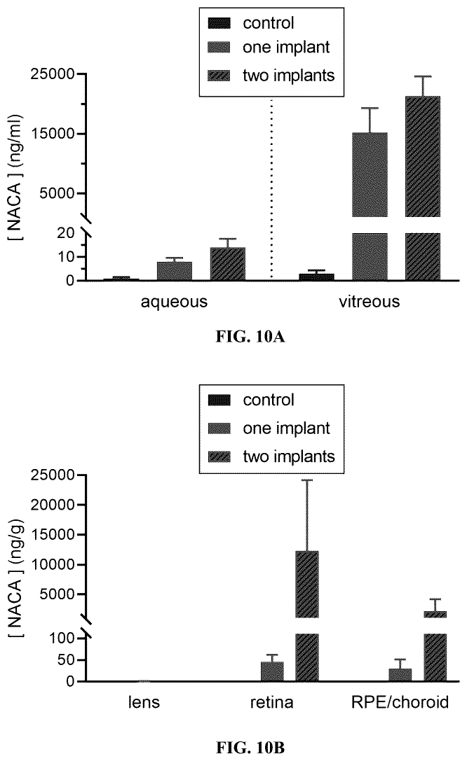

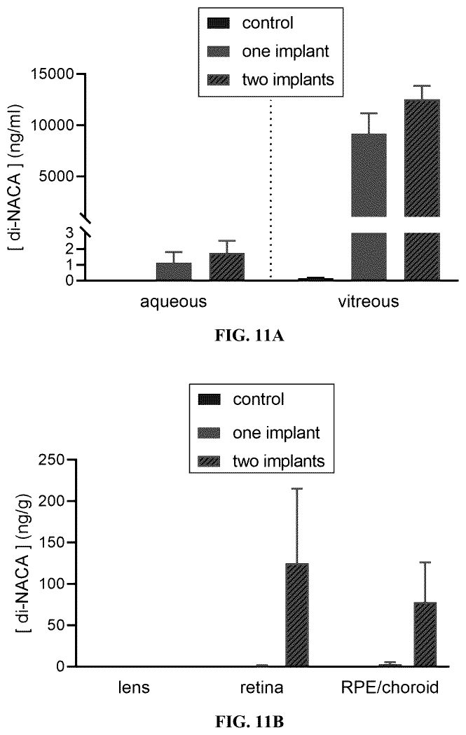

NACA: As shown in FIG. 10A, NACA was present at an extremely low level in the aqueous humor of the control group. In both treated groups, the concentration of NACA was elevated, but remained very low. In the vitreous humor, NACA was present at a very low level in the control group (approximately 3 ng/mL), but was dramatically higher in both treated groups (approximately 15000-20000 ng/mL).

As shown in FIG. 10B, NACA was undetectable in the lens samples in all three groups. In the retina, NACA was undetectable in the untreated group, and only modestly elevated in the single (fast-release) implant group. The disparity between the elevated vitreous level and low retinal level in the single implant group is surprising. In the dual implant group, NACA was, on average, highly elevated; however, there was great variability between samples, with one of the six samples displaying an extraordinarily high level (30-fold higher than the next highest sample, which was itself 25-fold higher than the remaining four samples). In the RPE/choroid, results displayed the same pattern as the retina: NACA was undetectable in the untreated group, and only modestly elevated in the single implant group, but was highly elevated in the dual implant group, albeit not to the magnitude measured in the corresponding retinal samples. Again, there was great variability between animals in the dual implant group, with the same two animals showing high and very high levels of NACA.

di-NACA: As shown in FIG. 11A, di-NACA was undetectable in the aqueous humor of the control group, while in both treated groups, the concentration of di-NACA was extremely low (approximately 1-2 ng/mL). In the vitreous humor, di-NACA was essentially undetectable in the control group (approximately 0.2 ng/mL), but was dramatically higher in both treated groups (approximately 9000-13000 ng/mL).

As shown in FIG. 9B, di-NACA was undetectable in the lens samples in all three groups. In the retina, di-NACA was undetectable in the untreated group, and essentially undetectable in the single implant group, but reasonably elevated in the dual implant group (125 ng/g tissue weight). The disparity between the high levels in the vitreous and low levels in the retina of both treated groups is surprising. In the RPE/choroid, results displayed the same pattern as the retina: di-NACA was undetectable in the untreated group, barely detectable in the single implant group, and modestly elevated in the dual implant group (78 ng/g tissue weight).

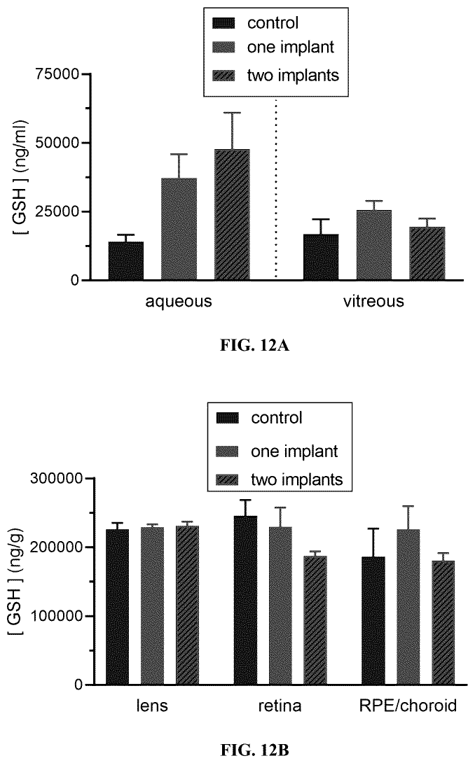

GSH: As shown in FIG. 12A, GSH was present at a high concentration in the aqueous humor of the control group (approximately 14000 ng/mL). This level of GSH equates to a molarity of 45 μM, which is broadly similar to the 25 μM value reported in the literature (Richer and Rose, 1998). The GSH concentration was higher in both the single and double implant groups (approximately 37000-47000 ng/mL). In the vitreous humor, GSH was present at a concentration of approximately 17000 ng/mL in the control group. The GSH concentration was somewhat higher in both the single and double implant groups (approximately 19000-25000 ng/mL).

As shown in FIG. 12B, GSH was present at a very high concentration in all three tissues (lens, retinal, RPE/choroid) in all three groups (approximately 20000 ng/g tissue weight). There was no pattern of GSH elevation in the single or dual implant groups in any tissue.

Summary. Levels of NAC, NACA and di-NACA were low in the aqueous humor of all groups. This is not particularly surprising given the rapid turnover of aqueous humor. It is, however, of interest that the GSH level in the aqueous humor was higher in both treated groups. In the lens, NAC, NACA and di-NACA were undetectable in all groups.

In the vitreous, the data indicate that implants containing NPI-002 (di-NACA) dramatically elevated vitreous humor levels of NACA and di-NACA when analyzed 3 months after surgery. Interestingly, while the levels of both compounds were higher in the double implant group, the differences between the single and double implant groups were not particularly marked. The elevated levels of di-NACA and NACA in the vitreous humor of the treated groups did not translate to statistically significantly higher levels of GSH.

In the RPE/choroid, the results for NAC, NACA, and di-NACA essentially mirrored those of the retina, but at slightly lower levels.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” issued to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only. Each of the composition of the present invention may comprise diNACA, may consist essentially of diNACA, or may consist of diNACA, as outlined hereinabove.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBB AAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refer condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organization cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Ahmad, et al., J. Amer. Chem. Soc. 101, 2669 (1979).
Beebe D C, Holekamp N M, Shui Y B. Oxidative damage and the prevention of age-related cataracts. Ophthalmic Res. 2010; 44(3):155-65. doi: 10.1159/000316481. Epub 2010 Sep. 9. PMID: 20829639; PMCID: PMC2952186.
Belin P J and Parke D W. Complications of vitreoretinal surgery. Curr Opin Ophthalmol 2020; 31, 167-173. doi: 10.1097/ICU.0000000000000652.
Campochiaro P A, Mir T A. The mechanism of cone cell death in Retinitis Pigmentosa. Prog Retin Eye Res. 2018 January; 62:24-37. doi: 10.1016/j.preteyeres.2017.08.004. Epub 2017 Sep. 27. PMID: 28962928.
Dilger R N, Baker D H. Oral N-acetyl-L-cysteine is a safe and effective precursor of cysteine. J Anim Sci. 2007 July; 85(7):1712-8. doi: 10.2527/jas.2006-835. Epub 2007 Mar. 19. PMID: 17371789.
FDA News Release “FDA approves novel gene therapy to treat patients with a rare form of inherited vision loss”, 2017, accessed 23 Jan. 2022 at: https://www.fda.gov/news-events/press-announcements/fda-approves-novel-gene-therapy-treat-patients-rare-form-inherited-vision-loss.
Frick K D, Joy S M, Wilson D A, Naidoo K S, Holden B A. The Global Burden of Potential Productivity Loss from Uncorrected Presbyopia. Ophthalmology. 2015 August; 122(8):1706-10. doi: 10.1016/j.ophtha.2015.04.014. PMID: 26190438.
Greenfield, et al., Cancer Res. 50, 6600-6607 (1990).
Heller, et al., Methods In Enzymology 112, 422-436 (1985).
Heller et al. Adv. Polymer Sci. 107, 41 (1993).
Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90.
Jaffe, G. J. et al., Journal of Ophthalmology and Vision Surgery, Vol 41, No. 11, October 2000.
Jeong, et al, Bioconj. Chem. 14, 473 (2003).
Izzotti A, Bagnis A, Sacca SC. The role of oxidative stress in glaucoma. Mutat Res. 2006 March; 612(2):105-14. doi: 10.1016/j.mrrev.2005.11.001. Epub 2006 Jan. 18. PMID: 16413223.
Lee S Y, Usui S, Zafar A B, Oveson B C, Jo Y J, Lu L, Masoudi S, Campochiaro PA. N-Acetylcysteine promotes long-term survival of cones in a model of retinitis pigmentosa. J Cell Physiol. 2011 July; 226(7):1843-9. doi: 10.1002/jcp.22508. PMID: 21506115.
Lim J C, Umapathy A, Donaldson P J. Tools to fight the cataract epidemic: A review of experimental animal models that mimic age related nuclear cataract. Exp Eye Res. 2016 April; 145:432-443. doi: 10.1016/j.exer.2015.09.007. Epub 2015 Sep. 25. PMID: 26391448.
LUXTURNA Prescribing Information, 2017 accessed 23 Jan. 2022 at: https://www.fda.gov/media/109906/download.
Petrash J M. Aging and age-related diseases of the ocular lens and vitreous body. Invest Ophthalmol Vis Sci. 2013 Dec. 13; 54(14):ORSF54-9. doi: 10.1167/iovs.13-12940. PMID: 24335070; PMCID: PMC3864378.
Prescott L F, Park J, Ballantyne A, Adriaenssens P, Proudfoot A T. Treatment of paracetamol (acetaminophen) poisoning with N-acetylcysteine. Lancet. 1977 Aug. 27; 2(8035):432-4. doi: 10.1016/s0140-6736(77)90612-2. PMID: 70646.
Rahil, et al, J. AM. CHEM. SOC. 103, 1723 (1981).
Richer S P and Rose R C (1998). Water soluble antioxidants in mammalian aqueous humor: interaction with UV B and hydrogen peroxide. Vision Research 38: 2881-2888.
Smilkstein M J, Knapp G L, Kulig K W, Rumack B H. Efficacy of oral N-acetylcysteine in the treatment of acetaminophen overdose. Analysis of the national multicenter study (1976 to 1985). N Engl J Med. 1988 Dec. 15; 319(24):1557-62. doi: PMID: 3059186.
Ster A M, Popp R A, Petrisor F M, Stan C, Pop V I. The Role of Oxidative Stress and Vascular Insufficiency in Primary Open Angle Glaucoma. Clujul Med. 2014; 87(3):143-6. doi: 10.15386/cjmed-295. Epub 2014 Aug. 5. PMID: 26528013; PMCID: PMC4508597.
Wall G M; Johnson D; Rubenstein A; Tucker R; Bolger J. Method for Preparation of N-Acetyl Cysteine Amide and Derivatives Thereof. U.S. Pat. No. 10,590,073B2 (United States), issue date 2020-03-17.
Wall G M. Prodrug for the treatment of disease and injury of oxidative stress. PCT/US21/14819. Filed 2021 Jan. 23.
Wall G M. Methods of Making Deuterium-Enriched N-acetylcysteine Amide (D-NACA) and (2R, 2R′)-3,3′-Disulfanediyl BIS(2-Acetamidopropanamide) (DINACA) and Using D-NACA and DINACA to Treat Diseases Involving Oxidative Stress. AU2018365900B2 (Australia), issue date 2020-06-18.
Wang G M, Raghavachari N, Lou M F. Relationship of protein-glutathione mixed disulfide and thioltransferase in H2O2-induced cataract in cultured pig lens. Exp Eye Res. 1997 May; 64(5):693-700. doi: 10.1006/exer.1996.0251. PMID: 9245898.
Zhu et al. Effect of antioxidant N-acetylcysteine on diabetic retinopathy and expression of VEGF and ICAM-1 from retinal blood vessels of diabetic rats. Molecular Biol Rep 2012; 39(4): 3727-3735.
U.S. Pat. No. 6,217,895
U.S. Pat. No. 6,548,078
U.S. Pat. Nos. 4,521,210; 4,853,224; 4,997,652; 5,164,188; 5,443,505; 5,501,856; 5,824,072; 5,869,079; 6,074,661; 6,331,313; 6,369,116; 6,699,493, and 6,726,918
U.S. application Ser. No. 10/966,764, filed Oct. 14, 2004; Ser. No. 11/039,192, filed Jan. 19, 2005; and 60/587,092, filed Jul. 12, 2004.

What is claimed is:

1. A biodegradable intraocular implant, comprising: (2R,2R′)-3,3′-disulfanediyl bis(2-acetamidopropanamide) (diNACA) in a pharmaceutically acceptable polymer.

2. The intraocular implant of claim 1, wherein the implant is selected from at least one of: about 0.1-0.5 mm in diameter and about 4-10 mm long, about 0.2-0.4 mm in diameter and about 5-8 mm long, about 0.3 mm in diameter and about 6-7 mm long, about 0.4 mm in diameter and about 7 mm long, or about 0.05 mm in diameter and between or about 5 to 8 mm long, or the implant comprises a plurality of openings or holes in the polymer, a coating, or both.

3. The intraocular implant of claim 1, wherein the pharmaceutically acceptable polymer is a poly (lactide-co-glycolide), polycarbamate polyurea, poly(vinyl ester), poly(methyl methacrylate), poly(vinyl chloride), polyamide, nylon, poly(ethylene terephthalate), rubber, silicone, polyisoprene, polyisobutylene, polybutadiene, polyethylene, polytetrafluoroethylene, poly(vinylidene chloride), polyacrylonitrile, polyvinylpyrrolidone, chlorinated polyethylene, polytrifluorochloroethylene, poly(ethylene chlorotrifluoroethylene), polytetrafluoroethylene, poly(ethylene tetrafluoroethylene), poly(4,4-isopropylidene diphenylene carbonate), polyurethane, polyperfluoroalkoxy, poly(vinylidene fluoride), vinylidene chloride-acrylonitrile copolymer, vinyl chloride-diethyl fumarate copolymer, silicone rubber, poly(dimethyl siloxanes), ethylene-propylene rubber, silicone-carbonate copolymers, vinylidene chloride-vinyl chloride copolymer, vinyl chloride-acrylonitrile copolymer, poly(olefins), poly(vinyl-olefin), poly(styrene), poly(halo-olefin), poly(vinyl) ester, poly(alkylacrylate), poly(oxides), poly(esters), polyamides, and polycarbonates, or mixtures thereof.

4. The intraocular implant of claim 1, wherein the diNACA is in the implant at an amount of at least between 21-65 weight percent (wt %), between 1-20 wt %, between 5-15 wt %, between 7-12 wt %, or about 10 wt %.

5. The intraocular implant of claim 1, wherein the intraocular implant comprises at least one of:
an antioxidant in the biodegradable polymer as a first intraocular implant; and an auxiliary agent associated with the biodegradable polymer as a second intraocular implant; wherein the antioxidant is present in the first intraocular implant in an amount of at least about 10 weight percent;
the implant is suitable for intravitreal placement in an eye of an individual, the antioxidant being present in an amount effective to treat an ocular condition of the eye; and an auxiliary agent associated with the biodegradable polymer as an intraocular implant structured for intravitreal placement in an eye of an individual, the auxiliary agent being present in an amount effective to reduce an occurrence of at least one undesired effect present upon otherwise identical administration of the antioxidant alone; or wherein the antioxidant is present in the intraocular implant in an amount of at least about 10 weight percent; or
further comprising an antioxidant selected from the group consisting of N-acetylcysteine (NAC), N-acetylcysteine amide (NACA), lipoic acid, lipoic acid choline ester, salts and mixtures thereof, and optionally an antioxidant auxiliary agent.

6. A biodegradable intraocular implant system comprising an antioxidant suitable for treatment of ophthalmic diseases or conditions involving oxidative stress in an animal or human.

7. The biodegradable intraocular implant system of claim 6, wherein the antioxidant comprises (2R,2R′)-3,3′-disulfanediyl bis(2-acetamidopropanamide) (diNACA) or the combination of (2R,2R′)-3,3′-disulfanediyl bis(2-acetamidopropanamide) (diNACA) and an auxiliary agent in a matrix of a polymer or in alternating layers.

8. The biodegradable intraocular implant system of claim 7, wherein the antioxidant comprises (2R,2R′)-3,3′-disulfanediylbis(2-acetamidopropanamide) (diNACA) or the diNACA and an auxiliary agent are comprised in the same implant or in different implants.

9. The biodegradable intraocular implant system of claim 8, wherein the antioxidant is in the implant system at an amount of at least between 21-65 weight percent (wt %), between 1-20 wt %, between 5-15 wt %, between 7-12 wt %, or about 10 wt %.

10. A method for treatment of ophthalmic oxidative stress in an animal or human subject that comprises:
identifying the animal or human patient in need of treatment for ophthalmic oxidative stress of an eye; and
administering to the animal or human patient a therapeutically effective amount of N-acetylcysteine amide (NACA) or (2R,2R′)-3,3′-disulfanediyl bis(2-acetamidopropanamide) (diNACA) in an intravitreal implant (diNACA implant).

11. The method of claim 10, wherein the ophthalmic oxidative stress of the eye is selected from at least one of cataracts, cataracts in a subject that does not have diabetes, corneal endothelial cell loss, age-related macular degeneration, presbyopia, retinitis pigmentosa (RP), Usher syndrome, Stargardt syndrome, glaucoma, diabetic retinopathy, or retinal disease.

12. The method of claim 10, wherein the diNACA is in a dosage form for a delivery route selected from the group consisting of intravitreal, intrastromal, intracameral, subtenon, retinal, subretinal, retrobulbar, peribulbar, suprachoroidal, subchoroidal, conjunctival, subconjunctival, episcleral, posterior juxtascleral, anterior juxtascleral, circumcorneal, topical, and tear duct, and the diNACA is provided in or with a pharmaceutically acceptable carrier.

13. The method of claim 10, wherein the diNACA is administered at least one of:
in daily doses of about 0.25, 1, 2, 3, 4, 5, 6, 7, 8, 9, 10, 20, 25, 30, 40, 45, 50, 60, 70, 75, 80, 90, 100, 110, 120, 125, 130, 140 to 150 mg/Kg;
in a single dose of about 1, 2, 3, 4, 5, 6, 7, 8, 9, 10, 20, 25, 30, 40, 45, 50, 60, 70, 75, 80, 90, 100, 110, 120, 125, 130, 140, 150, 175, 200, 250, 300, 400, 500, 600, 700, 750, 800 to 900 micrograms;
is administered once every 1, 2, 3, 4, 5, or 6 months;
a dose for administration is about 1, 10, 100, 150, 150, 300, 333, 400, 500, 600, 700, 750, 800, 900, 1,000, 2,500, 5,000, 7,500, or 10,000 micrograms per dose; or
the diNACA is in the implant at an amount of at least between 21-65 weight percent (wt %), between 1-20 wt %, between 5-15 wt %, between 7-12 wt %, or about 10 wt %.

14. The method of claim 10, wherein the diNACA is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, NAC, NACA, propyl gallate, α-tocopherol, citric acid, ethylenediamine tetraacetic acid (EDTA), sorbitol, tartaric acid, or phosphoric acid.

15. The method of claim 10, wherein the diNACA implant is at least one of: manually inserted into the eye, injected into the eye, breaks apart and dissolves or biodegrades in the eye, or is administered prophylactically to prevent ophthalmic disease or condition involving oxidative stress.

16. A nonbiodegradable, nonbioerodible or nonbioabsorbable intraocular implant, comprising: (2R,2R′)-3,3′-disulfanediyl bis(2-acetamidopropanamide) (diNACA) in a pharmaceutically acceptable polymer.

17. The implant of claim 16, wherein the antioxidant is selected from the group consisting of N-acetylcysteine, N-acetylcysteine amide, (2R,2R′)-3,3′-disulfanediyl bis(2-acetamidopropanamide) (diNACA), lipoic acid, lipoic acid choline ester, salts thereof, auxiliary agents, and mixtures thereof.

18. The implant of claim 16, wherein the implant is selected from at least one of:
a size of about 0.1-0.5 mm in diameter and about 4-10 mm long, about 0.2-0.4 mm in diameter and about 5-8 mm long, about 0.3 mm in diameter and about 6-7 mm long, about 0.4 mm in diameter and about 7 mm long, or about 0.05 mm in diameter and between or about 5 to 8 mm long, or the implant comprises a plurality of openings or holes in the polymer, a coating, or both; or
the diNACA is in the intraocular implant at an amount of at least between 21-65 weight percent (wt %), between 1-20 wt %, between 5-15 wt %, between 7-12 wt %, or about 10 wt %.

19. The implant of claim 16, wherein the polymer is a polycarbamate polyurea, poly(vinyl ester), poly(methyl methacrylate),poly(vinyl chloride), polyamide, nylon, polyethyleneterephthalate), rubber, silicone, polyisoprene, polyisobutylene, polybutadiene, polyethylene, polytetrafluoroethylene, poly(vinylidene chloride), polyacrylonitrile, polyvinylpyrrolidone, chlorinated polyethylene, polytrifluorochloroethylene, polyethylene chlorotrifluoroethylene, polytetrafluoroethylene, poly(ethylene tetrafluoroethylene), poly(4,4-isopropylidene diphenylene carbonate), polyurethane, polyperfluoroalkoxy, poly(vinylidene fluoride), vinylidene chloride-acrylonitrile copolymer, vinyl chloride-diethyl fumarate copolymer, silicone rubber, polydimethylsiloxanes), ethylene-propylene rubber, silicone-carbonate copolymers, vinylidene chloride-vinyl chloride copolymer, vinyl chloride-acrylonitrile copolymer, poly(olefins), poly(vinyl-olefin), poly(styrene), poly(halo-olefin), poly(vinyl ester), polyalkylacrylate, polyoxides, polyesters, polyamides, and polycarbonates, or mixtures thereof.

20. The implant of claim 16, wherein the diNACA implant is adapted for administration to the eye at a location selected from the group consisting of intravitreal, intrastromal, intracameral, subtenon, retinal, subretinal, retrobulbar, peribulbar, suprachoroidal, subchoroidal, conjunctival, subconjunctival, episcleral, posterior juxtascleral, anterior juxtascleral, circumcorneal, topical, and tear duct.

21. A method for treatment of cataracts, corneal endothelial cell loss, age-related macular degeneration, presbyopia, retinitis pigmentosa (RP), Usher syndrome, Stargardt syndrome, glaucoma, diabetic retinopathy, cataracts in a subject that does not have diabetes, or other retinal disease in an eye of an animal or human subject comprising:
obtaining an intravitreal implant (diNACA implant) comprising a therapeutically effective amount of N-acetylcysteine amide (NACA) or (2R,2R′)-3,3′-disulfanediyl bis(2-acetamidopropanamide) (diNACA); and
delivering to a pre-determined area in, at, or about the eye the diNACA implant that delivers a therapeutically effective amount of diNACA to the pre-determined area of the eye to treat cataracts, corneal endothelial cell loss, age-related macular degeneration, presbyopia, retinitis pigmentosa (RP), Usher syndrome, Stargardt syndrome, glaucoma, diabetic retinopathy, cataracts in a subject that does not have diabetes, or other retinal disease.

22. The method of claim 21, wherein the diNACA implant is administered to the pre-determined area of the eye selected from the group consisting of intravitreal, intrastromal, intracameral, subtenon, retinal, subretinal, retrobulbar, peribulbar, suprachoroidal, subchoroidal, conjunctival, subconjunctival, episcleral, posterior juxtascleral, anterior juxtascleral, circumcorneal, topical, and tear duct.

23. The method of claim 21, wherein the diNACA is administered at least one of:
in daily doses of about 0.25, 1, 2, 3, 4, 5, 6, 7, 8, 9, 10, 20, 25, 30, 40, 45, 50, 60, 70, 75, 80, 90, 100, 110, 120, 125, 130, 140 to 150 mg/Kg;
in a single dose of about 1, 2, 3, 4, 5, 6, 7, 8, 9, 10, 20, 25, 30, 40, 45, 50, 60, 70, 75, 80, 90, 100, 110, 120, 125, 130, 140, 150, 175, 200, 250, 300, 400, 500, 600, 700, 750, 800 to 900 micrograms;
is administered once every 1, 2, 3, 4, 5, or 6 months;
in the implant at an amount of at least between 21-65 weight percent (wt %), between 1-20 wt %, between 5-15 wt %, between 7-12 wt %, or about 10 wt %; or
is about 1, 10, 100, 150, 150, 300, 333, 400, 500, 600, 700, 750, 800, 900, 1,000, 2,500, 5,000, 7,500, or 10,000 micrograms per dose.

24. The method of claim 21, wherein the diNACA is administered with a second active agent selected from at least one of ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, NAC, NACA, propyl gallate, α-tocopherol, citric acid, ethylenediamine tetraacetic acid (EDTA), sorbitol, tartaric acid, or phosphoric acid.

25. The method of claim 21, wherein the diNACA implant is at least one of: manually inserted into the eye; the implant breaks apart and dissolves or biodegrades in the pre-determined area of the eye; or the diNACA is administered prophylactically to prevent ophthalmic disease or condition in or about the pre-determined area of the eye.
